(19) United States
(12) Patent Application Publication
(10) Pub. No.: US 2026/0241093 A1
(43) Pub. Date: Aug. 20, 2026

(54) DRUG DELIVERY SYSTEM

(71) Applicant: SHL Medical AG, Zug (CH)

(72) Inventors: Martin Michael Coyne, III, Boonton, NJ (US); Christopher James Franzese, Boonton, NJ (US); Jacob Leon Turetsky, Norwalk, CT (US); Yukiko Naoi, Norwalk, CT (US); Jeffrey Kapec, Norwalk, CT (US); Torbjörn Neby, Nacka Strand (SE); Stephen Allison Porter, Baltimore, MD (US); Mariano Mumpower, Baltimore, MD (US); Nils Weber, Zug (CH); James Thomas Hawthorne, Boonton, NJ (US)

(21) Appl. No.: 19/109,676

(22) PCT Filed: Sep. 8, 2023
(86) PCT No.: PCT/EP2023/074728

§ 371 Date: Mar. 7, 2025

Related U.S. Application Data
(60) Provisional application No. 63/405,062, filed on Sep. 9, 2022.
(60) Provisional application No. 63/455,427, filed on Mar. 29, 2023.

(30) Foreign Application Priority Data
Apr. 24, 2023 (EP) 23169499.3

(51) Int. Cl. A61M 5/14; A61M 5/168

(52) U.S. Cl. CPC A61M 5/1407; A61M 5/1413; A61M 5/16827; A61M 2205/3368

(57) ABSTRACT

The present disclosure relates to a drug delivery system comprising a drive unit. The drive unit comprises a drive unit interface, a drug interface, a pump assembly or a motor assembly configured to deliver a drug, a drug delivery controller and a power source. The drug delivery system further includes at least one drug unit connectable to the drive unit. The drug unit comprises a drug reservoir for storing a drug, an identification comprising information on the drug, a drug interface communicating with the drug reservoir, and a drug unit interface. The drug unit interface is configured to communicate with the drive unit interface. The drug interface of the drug unit is configured to communicate with the drug interface of the drive unit. The drug delivery system further includes a terminator connectable to the drug unit. The terminator unit comprises one or more components of a system controller, a terminator unit interface configured to communicate with the drug unit interface, and wherein the terminator unit is configured to close at least one of the drug unit interface and the drug interface of the drug unit.

### TECHNICAL FIELD

The present disclosure relates to a drug delivery system, configurable control system, a method for assembling a drug delivery system and a drug unit for use in a drug delivery system.

### BACKGROUND

Drug delivery is becoming more personal, for the benefit of both patients and health care providers (HCPs). Devices are enabling home delivery, and course parameters are being customized to the patient (drug types, volumes, sequences). Formerly, only intravenous (IV) delivery in a clinic allowed such complexity and customization. Accommodating this complexity with subcutaneous (SC) delivery of these medications is challenging. This new class of large-volume subcutaneous delivery devices must allow customization while not placing any burden on the patient to operate. Current at-home delivery devices either lack easy customization or require specialized training and costly equipment that patients may be uncomfortable with. Thus, solutions that allow broader patient access are advantageous. However, numerous problems must be solved before this may broadly take place.

### Problem #1: Medication and Regimen Complexity

Individual medications are often part of a larger regimen of medicines, with standardized regimens corresponding to a specific disease state, treatment regimen, or medication. In a clinic setting, order sets contain all the information required to administer a standardized regimen. For example, an oncology regimen might include pre-medications, oncology treatments, and post-medications, all contained in an order set. Existing drug delivery devices are designed to administer a single medication, limiting the ability to move therapy from the clinic to the home setting.

Furthermore, medication order sets may direct clinical staff to perform specific patient monitoring and permit contingent administration of emergency medication. This is particularly important for medications that cause infusion-related reactions in certain patients. Infusion reactions are potentially fatal, systemic reactions related to mode of action of the medication. Systemic infusion reactions are clinically distinct from localized injection site reactions or erythema from administration of a single agent such as would occur with an autoinjector, prefilled syringe, or OBI (on-body injection) device, which are uncomfortable but not life-threatening. They demand an immediate halt to medication administration and administration of one or more counteracting medications. However, prior art devices neither allow detection of systemic infusion reactions nor delivery of emergency medication and cannot be safely used to administer medications where systemic infusion reactions could occur. This is a particular concern for biologic therapies and is especially relevant to oncology treatments.

### Problem #2: Care Continuity & Safety: Regimens, EHR, Interlocks

Even if the challenges associated with variable medications and medication regimens are solved, the process by which medications may be provided and administered to patients remains problematic with the movement from IV to SC delivery and from in-clinic administration to the home.

In the clinic setting, Electronic Health Record (EHR) systems are now ubiquitous, and are used to manage patient care, particularly in complex disease states, such as oncology. Current standard-of-care IV delivery integrates smoothly with EHR and ordering systems and uses only a few standardized components, such as vials, syringes, and IV bags. Pharmacists, technicians, and nurses are responsible for the whole process and provide vital safety checks throughout.

In the clinic setting, administration of a medication regimen, associated monitoring, and clinical decision-making are documented in the patient's record within an electronic health record (EHR) system. The purpose of the EHR is to provide a complete clinical record of care for a patient, and safely manage medication regimens without relying on human memory or introducing human error. Healthcare providers update and review the EHR system in real-time for a given patient. Current drug delivery devices for home use do not have EHR interfaces, preventing their use with multi-medication regimens, contingent medication administration, or specific patient monitoring requirements. Moreover, administration of medication via other drug delivery devices, such as AIs (autoinjectors) & OBIs, may not be reflected in an EHR system. In the clinic setting, EHR systems also provide vital patient safety functions. EHR systems ensure patients may safely receive certain medications based on physical vital signs, laboratory testing values, or administration of prior medications as scheduled. However, prior art delivery devices used in the home setting are focused on a single medication, lack integration into EHR systems, and thus cannot provide safety interlocks that are present in the clinic. As a result, present devices cannot prevent administration of medications in unsafe conditions.

Programming: Medicament delivery systems can be designed to perform delivery of customized drug courses in the home, but that requires a means to communicate drug course parameters to the drive system. In the IV setting, IV pumps are customizable, but require a nurse with special training to program the pump at point-of-care.

### Problem #3: Rapid Technology Evolution

Connected delivery devices offer many safety, adherence, and supportive advantages to patients, and a variety of connected AI and OBI devices have been proposed. However, connecting large volume drug delivery devices generally presents several challenges, each creating new needs for improved solutions:
Regulatory Complexity: Regulatory pathways for traditional drug delivery systems can be so long that technology becomes obsolete before they reach market. Many prior art devices have been exposed to this risk, and improved solutions are needed that decouple the underlying connectivity functionality from the core device architecture (e.g., drive, controller, and reservoir(s)).
Ease of Lifecycle Management: Electronic components in a drug delivery device may become obsolete, unavailable, or more advanced, necessitating update or redesign of circuitry for electronic control or connectivity. This is particularly true for connectivity features and functions, as new communication technology is evolving quickly, and may be desirable to implement in future product generations. A component change in a fully integrated electronic drive system with connectivity may necessitate a much larger re-validation activity on the entire system. Thus, improved solutions are needed to abstract the system into logical components that inherently limit the extent of revalidation that must be completed if electronic components must be changed.

Improved solutions are also needed to specifically allow devices to be made selectively connected using a variety of technologies that may be adapted or evolved without extensive revalidation activity. Further, improved solutions are required to allow selective inclusion or exclusion of a specific connectivity feature or function for specific patients.

Current connected or “smart” devices, such as autoinjectors and prefilled syringes, all contain a single medication that is known at the time of manufacture.

Moreover, different markets in which a drug delivery system is commercialized may have different lifecycle needs at different points and may evolve unequally.

Further, different regions may have access to different technology or infrastructure. Improved solutions are needed to allow the same core drug delivery device to be easily regionalized to allow those communication approach(es) for each specific market without impact the core device.

### SUMMARY

The present invention has been made in view of the above problems and provides, inter alia, the following advantages.

Different medications used with the system may each have different volumes and/or viscosities. For instance, some medications may be fixed doses, while others may be variable doses that are patient specific. In other instances, there may be numerous medications, while in others, there may be fewer medications, complicating efficient manufacturing of a delivery device. Thus, the present disclosure provides an improved system and apparatus for administration that may flexibly deliver multiple medications using a single pump mechanism that does not require a priori knowledge of the number, volume, or viscosity of medications, and may be flexibly configured to deliver one or more medications in a desired sequence governed by a prescribed medication regimen.

Furthermore, the present disclosure provides an apparatus, systems and methods that allow integration with existing administration workflows, such as an EHR system, and may only allow administration of medications under safe conditions, replicating the safety measures at home that are currently present in clinic settings.

The present disclosure further allows a control system to be adapted to medications contained within a drug delivery system for a given patient or treatment regimen, especially when these factors are unknown a priori.

Also, a form of a drug delivery device or system impacts patient acceptance and commercial appeal thereof. While off-body pumps and on-body injectors are well known in the prior art, they each suffer from specific issues. Off-body pumps are large, heavy, and unwieldy, making activities of daily living difficult. On-body injectors are wearable, as with an adhesive patch, but particularly as drug delivery devices get larger, on-body injectors are increasingly uncomfortable, unwieldly, or interfere with daily activities. Better solutions are needed to allow near-body (e.g., over or under clothing) wear of more complex devices that have increased medication delivery flexibility.

Thus, in another aspect of the present disclosure, an improved and compact medicament delivery system comprising a single module of medication or a plurality of modules of medication is provided.

In an aspect of the present disclosure, a single drive unit can accommodate everything from a single cartridge up to multiple complex sequences of cartridges and volumes. It can be worn as a belt or across the body. A stack herein represents a modular system of daisy chained cartridges which allow for various sequences without additional infrastructure. Each patient gets a system that is only as complex as they need for a specific therapy or regimen.

In an embodiment, said system comprises a single cartridge, containing an entire regimen of one or more medications, a stack of cartridges, each containing one for more medications in the regimen, and sequential single cassettes, each containing one or more medications in the regimen.

Reusable drug delivery devices have advantages for sustainability and patient convenience. However, they may be used over a long period time with storage in between, as in the home, or may be used frequently across multiple patients, as in the clinic setting. Particularly in the clinic setting, healthcare-associated infections (HAIs) pose risks of increasing patient treatment intensity and cross-transmission. HAIs are associated with longer hospital stays, post-discharge readmission, and higher expenses. As a result, there is a desire to avoid contamination by microbes, viruses, or fungi that may affect sterility of the medications delivered with such a device.

In an aspect of the present disclosure, the above infection risks can be mitigated.

In an aspect of the present disclosure, at least one of the drive unit, the drug unit, or the terminator unit may be reusable or refillable, respectively.

The present disclosure relates to a drug delivery system comprising a drive unit, wherein the drive unit comprises a drive unit interface, a drug interface, a pump assembly or a motor assembly configured to deliver a drug, a drug delivery controller and a power source; at least one drug unit connectable to the drive unit, wherein the drug unit comprises a drug reservoir for storing a drug, an identification comprising information on the drug, a drug interface communicated with the drug reservoir, and a drug unit interface, wherein the drug unit interface is configured to communicate with the drive unit interface, wherein the drug interface of the drug unit is configured to communicate with the drive interface of the drive unit; and a terminator unit connectable to the drug unit, wherein the terminator unit comprises one or more components of a system controller, a terminator unit interface configured to communicate with the drug unit interface, and wherein the terminator unit is configured to close at least one of the drug unit interface and the drug interface of the drug unit.

Various embodiments may preferably implement the following features.

Preferably, the drive unit interface and the drug unit interface comprise at least one of a mechanical or electrical interface.

Preferably, the drug interface of the drive unit and the drug interface of the drug unit comprise at least one of a mechanical connection (for physical attachment), a sealed pneumatic connection (for pressured air), or an electrical connection (for power and communication).

Preferably, the system comprises a plurality of drug units.

Preferably, each of the plurality of drug units comprises identical drug interfaces and drug unit interfaces.

Preferably, the drug units are configured to be connected via the drug interface and the drug unit interface.

Preferably, the drug units are configured to be connected between the drive unit and the terminator unit.

Preferably, the drug units are configured to be connected between the drive unit and the terminator unit to form a stack.

Preferably, the drug units are configured to be connected between the drive unit and the terminator unit in an order corresponding to a planned order of administration to a patient.

Preferably, the units are configured to communicate via a wired or wireless connection, preferably via the electrical interfaces.

Preferably, the drug delivery controller is configured to control the pump assembly or motor assembly for delivering the drug to a patient.

Preferably, the drug delivery controller preferably comprises a non-volatile memory. Preferably, at least one of the drive unit, the drug unit or the terminator unit comprises at least one user interface element configured to control, indicate, or control and indicate a system status.

Preferably, the at least one interface element is at least one of a button, LED or screen.

Preferably, the system status indicates at least one of a correct connection of the units, sequence of the units, information on expiry of the drug, battery level, dispense parameter information, dispense error information, dispense readiness information, dispense status information, connection status, patient clinical characteristics, occlusion status of a tubing set, or needle used with the system.

Preferably, at least one of the drug unit or the terminator unit comprises a wireless or wired connection module configured to communicate with at least one of a server, the internet or a terminal.

Preferably, the wireless or wired connection module is configured to receive and transmit drug delivery information.

Preferably, the drug delivery information is received or transmitted during filling of the drug reservoir within the drug unit.

Preferably, the drug delivery information corresponds to one or more aspects contained in an electronic health record system for a patient using the drug delivery system.

Preferably, the drug unit further comprises a temperature sensor configured to sense at least one of a temperature of a drug stored in the drug reservoir or an ambient temperature where the drug delivery system is used.

Preferably, the identification is stored in an electronic memory, an RFID tag, a 2D barcode, a 3D barcode, a data matrix, or a QR code.

Preferably, the identification comprises information on at least one of a medication contained a drug unit, a volume of the drug reservoir, volume of medication within the drug reservoir, drug delivery information, dispense parameters of the drug, flow rate of the drug, drug lot number, patient identifier, an identifier of a medication in a sequence of one or more medications, expiration date, a temperature condition of the drug, administration date, administration time, medication cycle number, serialization/identifier information for one or more of the medication, or drug unit.

Preferably, the system controller is configured to read the identification and communicate with the drug delivery controller to perform drug delivery according to the identification.

Preferably, one or more components of the system controller is reconfigurable and/or programable.

Preferably, the terminator unit is reusable or disposable.

The disclosure further relates to a method for assembling a system according to one of the preceding clauses, the method comprising filling at least one drug into the drug reservoir of at least one drug unit prior to assembly of the system, assembling the drug delivery system by connecting the drive unit, at least one drug unit and the terminator unit, and reading, by the system controller, the identification of the at least one drug unit.

The disclosure further relates to a drug unit for use in a system as described above, wherein the drug unit comprises a drug reservoir for storing a drug, an identification comprising information on the drug, a drug interface communicated with the drug reservoir, and a drug unit interface, and wherein the drug unit is configured to be connectable to a drive unit and a terminator unit.

The present disclosure further relates to a drug delivery system comprising a pump assembly configured to deliver a drug; at least one drug unit, wherein the drug unit comprises a drug reservoir for storing a drug and being connectable to the pump assembly, and at least one shell configured to receive the pump assembly and the drug reservoir.

Various embodiments may preferably implement the following features.

Preferably, the drug unit comprises two shells connected by a hinge or snap fit, wherein the pump assembly and the drug reservoir are provided between the shells.

Preferably, the drug unit comprises a medicament container frame and the drug reservoir and the pump assembly are provided on the medicament container frame.

Preferably, the medicament container frame is received in the shell.

Preferably, the drug unit further comprises a filter configured to filter a drug.

Preferably, the drug unit further comprises a tube frame configured to receive a flexible tube.

Preferably, the drug unit comprises a connection track and/or a connection feature, wherein the connection track and/or connection feature is/are configured to attach drug units to each other.

Preferably, the connection track and/or connection feature is/are provided on the shell.

Preferably, the drug unit comprises a communication interface, wherein the communication interface is provided on the shell.

Preferably, the system further comprises a drive unit, wherein the drive unit comprises a drug delivery controller and a power source.

Preferably, the drive unit comprises the pump assembly, and the drive unit is provided in the at least one shell.

Preferably, the drive unit comprises a connection feature and a communication interface.

Preferably, the drive unit is configured to be separably attached to a connection track of the drug unit.

Preferably, the communication interface is configured to communicate with a communication interface of the attached drug unit.

Preferably, the drug unit comprises a belt and/or a strap and/or a clip for attaching the drug unit to a patient.

Preferably, the belt and/or strap and/or clip is coated with a material having antimicrobial and/or antifungal and/or antiviral properties.

Preferably, the belt and/or strap and/or clip is at least partially formed of a material comprising antimicrobial and/or antifungal and/or antiviral properties.

Preferably, the drug unit comprises a removable programming key. Preferably, at least one drug unit is connected to the drive unit. Preferably, the removable programming key is connected to the drive unit.

Preferably, each removable programming key comprises connectors configured to connect a plurality of programming keys to each other.

Preferably, each programming key comprises internal logic and/or memory configured to communicate with the drive unit and/or configured to control drug delivery.

Preferably, the drug unit interface is the removable programming key.

Preferably, the identification carries at least one of type of drug, temperature, flowrate, or sequence information. Preferably, the drive unit is configured to read the information carried by the identification.

Preferably, the drive unit is configured to provide, based on the read information, a virtual stack of drug units indicating a sequence of drug delivery.

Preferably, the drive unit comprises at least one indicator configured to provide visual feedback on the virtual stack to a patient.

Preferably, the at least one indicator is one or more light emitting diodes, LEDs, and/or a display.

Preferably, the indicator is configured to output a warning or an error state relating to the virtual stack. Preferably, the warning or error state is output by emitting light, emitting light of different colours and/or emitting flashing light.

Preferably, the drive unit comprises at least one button, wherein the at least one button is configured to manage the virtual stack.

Preferably, managing the virtual stack comprises adding or deleting drug units and/or terminating addition or deletion of drug units and/or initiating drug delivery.

Preferably, the identification is a passive component. Preferably, the passive component is an RFID chip or near field communication, NFC, chip.

### BRIEF DESCRIPTION OF THE DRAWINGS

The present disclosure will be further described with reference to the appended figures.

FIG. 1 shows a drug delivery system according to an embodiment of the disclosure.

FIG. 2 shows a plurality of drug units and a terminator unit being connected according to an embodiment.

FIG. 3 shows a schematic diagram of a system according to an embodiment.

FIGS. 4A to 4E show a drug delivery system according to an embodiment of the disclosure.

FIG. 5 shows a drug delivery system according to an embodiment comprising a plurality of drug units as shown in FIGS. 4B and 4C.

FIG. 6 shows a drug delivery system according to an embodiment comprising removable programming keys.

### DETAILED DESCRIPTION

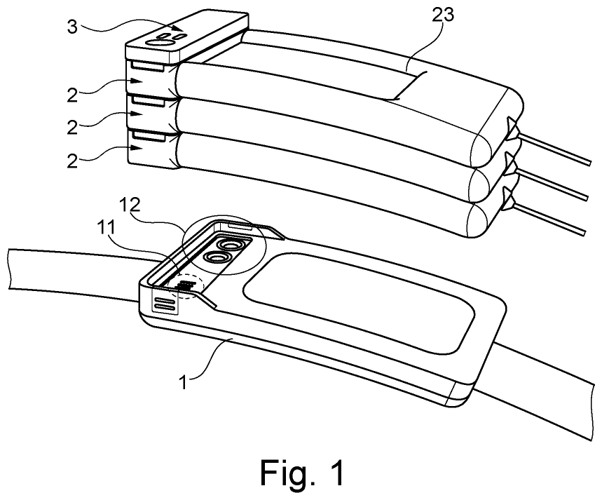

According to FIG. 1, the drug delivery system comprises a drive unit 1, a drug unit 2 and a terminator unit 3. The terms unit and module may be used interchangeably. Also, the terms drug and medicament may be used synonymously. Moreover, “and/or” and “at least one of” may denote that at least one element of the list may be present in the embodiment and thus also used synonymously.

The term “separable” in accordance with the present invention means that the drive unit 1 (pump unit) can be physically separated from the drug unit 2 (cassette). A separable drive unit 1 can also be carried on or affixed to the drug unit 2.

The term “independent” in accordance with certain embodiments of the present invention means that the drive unit 1 is functionally independent from the drug unit 2 and from the medicament bag(s). The drive unit 1 according to the present invention can thus co-operate with a plurality of different drug units 2, as desired by the user, wherein the drug unit 2 is adapted to communicate with a drive unit 1. Thus, the user is provided with several options as regards the use of the drive unit 1. If desired, however, the independence of the drive unit 1 can be deactivated or disabled or at least restricted so that a given drive unit 1 is operable with only one or a limited number of drug units 2.

The drug delivery system may be connected to at least one infusion set for delivering the medicament to the patient. One infusion set for each drug unit 2 may be provided. The infusion set may comprise a (flexible) tube and a needle corresponding to the type of drug and intended delivery.

The drive unit comprises a drive unit interface 11, a drug interface 12 and a pump assembly or a motor assembly 13. The pump assembly or a motor assembly 13 is configured to deliver a drug. Moreover, the drug unit comprises a drug delivery controller 14 and a power source 15. The power source 15 may be connected to each of the pump or motor assembly 13 and the drug delivery controller 14.

The drive unit 1 may be connected to a patient's body via a strap, an elastic band or similar.

The drug unit 2 is connectable to the drive unit 1. In FIG. 1, three drug units 2 are shown. This, however, is not to be understood as limiting and any number of drug units 2 may be provided, for example two, four, or five.

Each of the drug units 2 comprises a drug reservoir 23 for storing a drug, an identification 24 comprising information on the drug, a drug interface 22 communicated with the drug reservoir 23, a drug outlet 25, and a drug unit interface 21. The drug unit interface 21 is configured to communicate with the drive unit interface 11 and the drug interface 22 of the drug unit 2 is configured to communicate with the drug interface 12 of the drive unit 1.

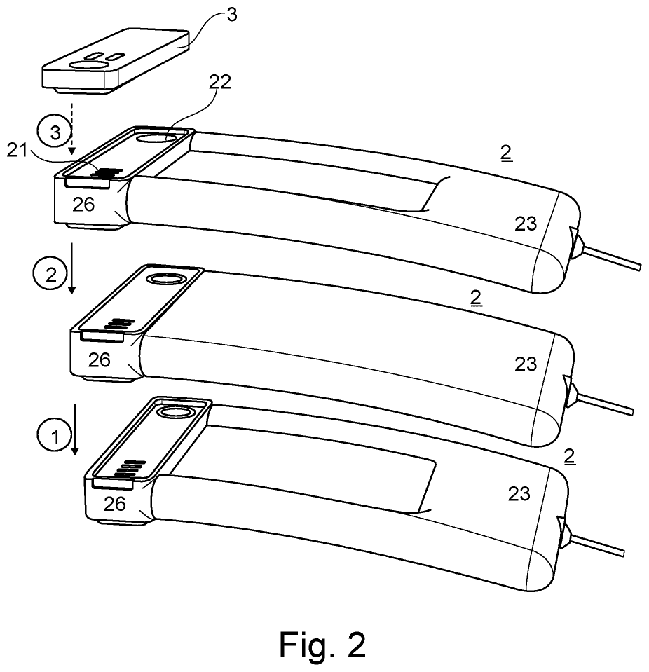

As can be seen from FIG. 1, the drug unit 2 may comprise a base part 26 in which the drug unit interface 21 and the drug interface 22 are provided and to which the drug reservoir 23 is attached and protrudes from. However, the drug unit 2 may also be integrally formed. In case a plurality of drug units 2 are provided as shown in FIG. 2, the drug units 2 may be configured to be connectable via their respective drug unit interfaces 21. That is, the drug unit interface 21 of the drug units 2 may preferably be accessible from two opposing sides of the base part 26 (cf. FIG. 2). Moreover, the plurality of drug units 2 may be configured to be connectable via their respective drug interfaces 22.

The terminator unit 3 is configured to be connectable to the drug unit 2. In case a plurality of drug units 2 are provided, the terminator unit 3 may be connected to the one located the farthest from the drive unit 1 (cf. FIG. 1).

The terminator unit 3 comprises one or more components of a system controller 34 and a terminator unit interface 31 configured to communicate with the drug unit interface 21. The terminator unit 3 is configured to close at least one of the drug unit interface 21 and the drug interface 22 of the drug unit 2. In particular, the drug unit interface 21 may be closed by the terminator unit interface 31. Closing may refer to sealing of the components, i.e. preventing liquids and gases from exiting or entering and preventing dust or other foreign substances from entering the drug unit(s) 2 and thus the drive unit 1.

The terminator unit 3 may be reusable or disposable.

The system controller 34 or components thereof may be connected to the drug delivery controller 14 via the terminator unit interface 31, the drug unit interface 21 and the drive unit interface 11. Although only components of the system controller 34 may be provided in the terminator unit 3, it may be referred to the “system controller 34” in the following for reasons of simplicity.

The drive unit interface 11 and the drug unit interface 21 may comprise a mechanical and/or electrical interface. Similarly, the terminator unit interface 31 may comprise a mechanical and/or electrical interface or a means to close the drug unit interface 21. The drug interface 12 of the drive unit 1 and the drug interface 22 of the drug unit 2 may comprise a sealed pneumatic connection and/or an optical connection.

As noted above, the system may comprise a plurality of drug units 2. Each of the plurality of drug units 2 comprises identical drug interfaces 22 and drug unit interfaces 21 on two opposing sides of the drug unit 2. The drug units 2 are configured to be connected via the drug interface 22 and the drug unit interface 21 and the drug units 2 are configured to be connected between the drive unit 1 and the terminator unit 3. The units may be connected to form a stack as shown in FIG. 1 or 2. The drug units 2 may be connected between the drive unit 1 and the terminator unit 3 in an order corresponding to a planned order of administration to a patient. However, the drug units 2 (and the drive unit 1 and terminator unit 3) may also be connected by other means, such as tubes and/or cables to provide communication (e.g. electronic and/or fluid and/or optical) between the units. In that manner, the units 1, 2, 3 do not have to be stacked but can also be physically distributed, e.g. on a patient's body.

In addition to the interfaces 11, 21, 31 and 12, 22, 32, a (releasable) mechanical connection such as a snap fit may be provided to connect the units 1, 2, 3 and hold them in place. The mechanical connection may also be integrated in one of the interfaces 11, 21, 31 and 12, 22, 32.

The units 1, 2, 3 may be configured to communicate via a wired or wireless connection, preferably via the electrical interfaces, i.e. the drive unit interface 11, the drug unit interface 21 and the terminator unit interface 31.

The drug delivery controller 14 may be configured to control the pump or motor assembly 13 for delivering the drug to a patient. The drug delivery controller 14 may comprise a non-volatile memory.

The drive unit 1 and/or the drug unit 2 and/or the terminator unit 3 may comprise at least one user interface element configured to control and/or indicate a system status.

The at least one interface element may be a button and/or LED and/or screen. The system status may indicate a correct connection of the units and/or sequence of the units and information on expiry of the drug and/or battery level and/or dispense parameter information and/or dispense error information and/or dispense readiness information and/or dispense status information and/or connection status and/or patient clinical characteristics and/or occlusion status of a tubing set (i.e. an occlusion alarm such as a global occlusion alarm indicator to help the patient/HCP localise the occlusion to a specific drug unit) and/or needle used with the system.

The drug unit 2 and/or the terminator unit 3 may comprise a wireless or wired connection module configured to communicate with a server and/or the internet and/or a terminal. The wireless or wired connection module may be configured to receive and transmit drug delivery information.

The drug delivery information may be received or transmitted during filling of the drug reservoir within the drug unit, e.g. in a factory or pharmacy. The drug delivery information may correspond to one or more aspects contained in an electronic health record system for a patient using the drug delivery system.

The drug unit may further comprise a temperature sensor configured to sense a temperature of a drug stored in the drug reservoir 23 and/or an ambient temperature where the drug delivery system is used.

The identification 24 may be stored in at least one of a memory, an RFID tag, a 2D/3D barcode, a data matrix, a QR code or similar.

The identification 24 preferably comprises information on the medication contained in a drug unit 2 (i.e. the respective drug reservoir 23) and/or a volume of the drug reservoir 23 and/or volume of medication within the drug reservoir 23 and/or drug delivery information and/or dispense parameters of the drug and/or flow rate of the drug and/or drug lot number and/or patient identifier and/or an identifier of a medication in a sequence of one or more medications and/or expiration date and/or a temperature condition of the drug and/or administration date and/or administration time and/or medication cycle number and/or serialization/identifier information for one or more of the medication and/or drug unit 2. A medication cycle herein may e.g. refer to the number associated with a therapy delivered in cycles (e.g., oncology therapy on weeks 1, 3, 5, 7, 9) or ordinal number (1st cycle, 2nd cycle, 3rd cycle).

The term drug delivery information, however, may encompass various parameters of the drug dispensing such as drug volume, reservoir volume, dispense parameters, flow rate, medication sequence, temperature condition, administration time, administration date, administration speed, medication cycle, etc.

The system controller 34 may be configured to read the identification 24 and to communicate with the drug delivery controller 14 to perform drug delivery according to the identification 24.

One or more components of the system controller 34 may be reconfigurable and/or programable. The terminator unit 3 and/or the drug unit(s) 2 may be disposable. The drive unit 1 may be reusable.

The present disclosure further relates to a method for assembling a system as described above. The method comprises filling at least one drug into the drug reservoir 23 of at least one drug unit 2 prior to assembly of the system, assembling the drug delivery system by connecting the drive unit 1, at least one drug unit 2 and the terminator unit 3 and reading, by the system controller 34, the identification 24 of the at least one drug unit 2.

The method may also comprise reading, by the system controller, an aspect of the configuration of the at least one drug unit.

The assembly of the drug units 2 and the terminator unit 3 is shown in FIG. 2 (drive unit 1 not shown), the assembled device is depicted in FIG. 1. In FIG. 2, the encircled numerals denote an exemplary order of assembly, the arrows the direction of assembly. The present disclosure also relates to a drug unit 2 for use in a system as described above. The drug unit 2 comprises a drug reservoir 23 for storing a drug, an identification 24 comprising information on the drug, a drug interface 22 communicated with the drug reservoir 23, and a drug unit interface 21. The drug unit 2 is configured to be connectable to a drive unit 1 and a terminator unit 3.

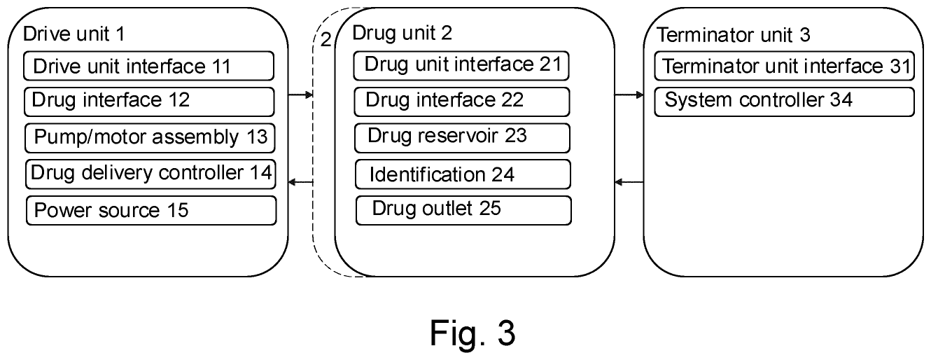

FIG. 3 provides a schematic overview of a system for medicament/drug delivery according to an embodiment. The dotted line indicates that one or more drug units 2 may be used when assembling the drug delivery system. The drive unit 2 and the drug unit(s) 2, the drug unit(s) among themselves as well as the drug unit(s) 2 and the terminator unit 3 may communicated with each other as indicated by the arrows. Communication may particularly be performed via the unit interfaces 11, 21, 31 and fluid communication, i.e. medicament communication, may be performed via the drug interfaces 12, 22. The drive unit 1 is thus able to communicate with the terminator unit 3 via the drug unit(s) 2.

The drive unit 1, the drug unit 2, the terminator unit 3 as well as the interfaces will now be described using exemplary embodiments. All elements and features described herein are compatible with each other.

Each unit 1, 2, 3 is provided with one or more of a mechanical, pneumatic, optical, or electrical connection, i.e. the interfaces 11, 21, 31 and 12, 22, 32. Each unit 1, 2, 3 has the same connection points in a predetermined location based on the form factor of the unit, even though each unit may serve different purposes.

Electrical connections include power distributed from the drive unit 1 to the rest of the assembled drug units 2 and the terminator unit 3. Electrical or optical connections also may form a communication bus allowing unidirectional or bidirectional communication between the drive unit 1 and other assembled drug units 2 and terminator unit 3. Connections and the communication bus provided may also be optionally used to provide user feedback as to the state of the device on one or more of the system's components (e.g., the drive unit 1, individual drug unit(s) 2, or the terminator unit 3).

The placement, number, and type of connectors may be widely varied, based on the configuration of the delivery device, types and characteristics of medications to be delivered, and connectors needed, as shown representatively in FIG. 1. The electrical common interface connection may be wired or wireless.

In FIG. 1, two pneumatic connectors are shown as an example, but the present disclosure is not limited thereto.

The drive unit 1 is an independent piece of the apparatus. For instance, the drive unit 1 may be a mass-produced, reusable unit for use in the home or clinic setting that has no history or pre-configuring with other components in the product ecosystem until they are physically connected at the time of delivery. Notably, the drive unit 1 is entirely agnostic to the number, quantity, or order of medications in the assembled unit.

The drive unit 1 contains a common mechanical and/or electrical interface 11 to the connected drug units 2, a type of pump or motor (assembly) 13 used to deliver a medication, logical and memory circuitry (drug delivery controller 14) necessary to control the pump or motor 13, and a battery (power source 15) to supply electrical power to the system (including connected units 2, 3, if necessary). The drive unit 1 may also contain user interface elements for control and indication of system status, such as buttons and LEDs. The mechanical interface between the drive unit and downstream units may include both a physical attachment mechanism and a sealed pneumatic connection.

The electrical interface between the drive unit and downstream units may be either wired, using some number of physical electrical connections, or wireless, relying on proximity between the drive unit 1 and other units at some point during the product workflow. The drive unit 1 is the source of power for the system in either implementation. In either case, the electrical interface is how information about the drug course is transferred from the drug units 2 and terminator unit 3 to the drive unit 1.

The logical and memory circuitry (which may be included in the drug delivery controller 14) in the drive unit 1 contains the sensing infrastructure and firmware necessary to deliver a drug course based on a set of parameters, particularly by controlling the pump or motor 13 and other associated components. The drive unit 1 does not natively contain those parameters-they are communicated to the drive unit 1 upon connection of the drug units 2 and terminator unit 3 and stored in its memory only during the delivery of that particular drug course.

The physical size of the drive unit 1 may change to accommodate pumps and/or batteries of different sizes. This allows the system to accommodate varying time and power requirements with scaled unit cost.

Drug units 2, in particular the drug reservoirs 23, may be either prefilled or pharmacy-filled, in any number, and in any combination. Different variations of each may exist and may be selected by a dispensing pharmacy in accordance with the prescription or medication order for a specific patient. The drug units 2 may be pharma-filled, as in the case of a fixed dose of a single medication, or a fixed-dose combination (FDC) of multiple medications. The medication information (such as volume, delivery parameters, etc., see above) may be stored in the identification 24.

Alternatively, drug units 2 may be pharmacy filled, for instance, with a variable volume or one of many different medications which are not available prefilled by pharma.

Notably, the parameters of each drug unit 2 need not be known a priori by the drive unit 1. Preferably, drug units 2 are mass-produced and disposable, and may optionally be made available in a variety of sizes (e.g., small, medium, large, or 10, 50, 100mL). Drug units 2 may have different internal primary containers (e.g., COC/Aclar film, as with known flexible bags, or EVA) within the same system. Pharmacy-filled drug units 2 may be stocked “off the shelf” empty by pharmacy and filled with any drug in any volume, and store only serial number and SKU information. Pharma filled drug units 2 may be provided with additional identifying information beyond those present in the pharmacy-filled versions, as discussed below (“Data Parameters”).

The drug unit 2 may simply be a fluid vessel with a common mechanical interface allowing it to connect to other drug units 2, the drive unit 1, and/or the terminator unit 3. Connection may also be provided via the drug unit interface 21 and/or the drug interface 22.

The drug unit 2 may also contain a common electrical interface (e.g. the interface 21) allowing it to connect to other units 1, 2, 3 and logical and memory circuitry necessary to store information and facilitate communication between connected units 1, 2, 3. The drug unit 2 may also contain user interface elements for indication of system status, such as LEDs, and/or sensing infrastructure, such as for drug temperature.

The unit electronics allow system error detection of counterfeit parts, incorrect number of units, incorrect unit order, and unexpected serial number. With the appropriate sensor, drug temperature errors at the time of dispense could also be detected. Depending on the required capabilities of the system, drug units 2 do not necessarily need electronics.

Assuming electronics are included, unfilled drug units stocked by pharmacies may contain only a serial number and SKU information identifying the product attributes.

That basic information could be used to build up a record of information in electronic health record systems and provide additional safeguards to proper device programming by the pharmacist. Even when filled, these drug units 2 would not necessarily store information about the filled drug-that information would reside in the terminator unit.

Prefilled drug units may contain the information above along with dispense parameters (volume, flow rate, etc.) and other information (drug lot numbers, expiration dates, etc.) programmed at the time of filling by the pharma company. They may be programmed similarly to the terminator unit using a wired or wireless interface to a factory computer.

The drug units 2 may not contain a power source. Any power required is provided by the drive unit 1 or by dedicated hardware in the dispensing pharmacy.

Tamper protection may be included in the drug units 2 in the form of single-use electrical, mechanical, or pneumatic connections. After initial connection of units, any subsequent disconnection would render the units inoperable by permanently breaking one or more of the previously mentioned connections.

Depending on the system architecture, the drug unit electronics may communicate with connected units 1, 2, 3 via a wired or wireless interface. In either case, one of several communication protocols could be used.

The terminator unit 3 is a disposable programming module manipulated by pharmacist at time of dispense to contain course parameters. That is, the patient may receive a pre-programmed terminator unit 3 corresponding to his/her medication and the assembled drug unit(s) 2. At one end of the drug delivery system, the terminator provides safe, consistent closure of the common interfaces (whether mechanically, optically, pneumatically, electrically, or otherwise) and, if provided in a physical embodiment, a visual indication to a patient or dispensing clinician the product is fully assembled (i.e. the drive unit 1, the drug unit(s) 2 and the terminator unit 3 are securely connected and closed/sealed). Alternatively, the terminator unit 3 may also be used as a “forcing function” to ensure that the system is appropriately assembled.

As a separate component of the system, the terminator unit's 3 electronics can be produced in different configurations to support various methods of programming info entry, including, by way of example and not limitation:
Physical knobs or switches that represent dispense parameter information (medication information), manipulated by a pharmacist during order fulfilment. These features may also be included on the individual drug units if desired, which is particularly advantageous if an unknown number of drugs will be used with the system.
Data transfer from pharmacy electronic order management system (e.g., electronic health record system/EHR) via a communication interface between a pharmacy device and a terminator unit 3.
Wireless/cloud data transfer from electronic health record system (EHR) via combination of a patient home network devices and/or a terminator unit's 3 wireless hardware modules. This could be effectuated for instance by NFC, cellular/wifi wall hub, mesh network, Bluetooth, through direct-to-cellular technology, or other suitable data transfer mechanism.

In the following, example scenarios and applications are presented.

Drug unit(s) 2 and terminator unit (components) 3 may be stocked by a pharmacy, then filled with medication (if needed), programmed, and assembled in accordance with a medication order for a specific patient as follows:
1. Pharmacist selects appropriate unfilled drug units from inventory. Each drug unit 2 is labelled with a barcode containing serial number and stock keeping unit (SKU) information. This corresponds to the identification 24. If the drug unit 2 includes electronics, the internal non-volatile memory contains the same information.
2. The pharmacist scans the barcode on each drug unit 2, creating a record in the computer system of the components used.
3. The pharmacist fills the drug units 2 with the required drug.
4. The pharmacist selects the appropriate terminator unit 3 and programs it. Depending on the type of terminator unit 3 selected, this action takes different forms
   a. The pharmacist reads course parameters from the health record and manually manipulates features on the terminator unit 3 to store them.
   b. The information from the interface is electronically stored in the terminator unit 3 when a wired or wireless connection is made.
   c. The terminator unit 3 receives parameters via a cloud interface upon connection to the drive unit 1 at the patient home. This terminator unit 3 may be reused by the patient.
5. The pharmacist assembles the drug unit(s) 2 and terminator unit 3 and signs off on the completed course before shipping it to the patient.

Thus, in a level 1 terminator unit 3, there is no special pharmacy equipment or connectivity needed and a mechanical setting of flow rate, time delays, etc. is performed.

In a level 2 terminator unit 3, flow rate, time delays, etc. are programmed e.g. via RFID (or similar) and a memory for drug and regimen information is provided.

In a level 3 terminator unit 3, a memory for drug, regimen and patient information is provided and an information exchange is performed via cloud integration (e.g. via EHR) without change to the core system.

The drive unit 1, as it may be reusable, may already be in possession of the patient. As explained above, the drive unit is agnostic to the medication and receives and processes data according to the attached drug unit(s) 2 and terminator unit 3. The drive unit 1 does therefore not need to be replaced or changed according to the medication.

In this context, reference is again made to FIG. 2 representing the assembly condition, e.g. in a pharmacy. The circled digits indicate the order of assembly for a three-medication regimen as the system is built by a dispensing pharmacist using three drug units 2 and ended with the terminator unit 3.

The assembled components together contain the information necessary to dispense the drug course when the assembly is connected to the drive unit 1 at the patient's home. Although the assembly contains electronics, all information is stored in non-volatile memory that does not require a continuous power source. The units 1, 2, 3 remain unpowered between programming at the pharmacy and connection to the drive unit 1 by the patient.

Communication between units can happen via wired or wireless communication.

When using a wired interface, a shift register may be used to describe the movement of information from the drug unit(s) 2 and terminator unit 3 to the drive unit 1. A shift register moves information in a continuous chain. In this case, the drug unit(s) 2 and terminator units 3 together make up a shift register. When commanded by the drive unit 1, the connected drug unit(s) 2 and terminator unit 3 pass course information to the adjacent unit. Each piece of information passes through each member of the assembled system. When the information reaches the drive unit 1 it is stored in memory for use during the drug dispense.

The shift register architecture is serial, meaning the physical order of the drug units 2 is preserved in the order in which information arrives at the drive unit 1. Each additional unit in the system includes its relevant information in the broader shift register, so the drive unit 1 has an awareness of exactly what is connected to it and in which order. Shift register-style serial communication may be implemented with one of several standard communication protocols (Serial Peripheral Interface (SPI), Universal Asynchronous Receiver/Transmitter (UART)) or via a custom protocol.

Wireless communication via some form of RFID between units 1, 2, 3 may be used instead. Proximity between units 1, 2, 3 must be enforced for communication to happen, but communication does not need to be continuous. A brief interaction before the dispense allows all information from the connected units 1, 2, 3 to be transmitted and stored in the drive unit.

With the apparatus components and principle of operation described, representative scenarios will be used to explain the principle of operation at a detailed level. While several medication administration scenarios are used in an illustrative manner, the present disclosure should not be limited to the scenarios detailed. In fact, the primary advantage of the common interface provided by the present invention is enablement of independent changes to any component part, fully abstracting the system components and allowing many different permutations-in fact, any desired permutation of components. The common interface 11, 21, 31 consists of mating connections and supporting electronics, such as logic and memory elements, that allow all units connected in the system to communicate information. As every member of the device ecosystem shares this common interface, any permutation of components can be made.

A serial protocol is not necessarily required-addressable protocols (such as inter-integrated circuit, I2C) may be used with extra features that preserve the information about the physical ordering of the drug units.

A few of the variables that can be accommodated by this system are listed include, by way of example but not limitation:
Any number of drug units 2 can be supported by this device, from one drug unit 2 up to the maximum within the capability of the drive unit 1
Drug units 2 may come in different sizes
Drug units 2 may be filled up to their maximum volume or some fraction of the maximum Drugs may be delivered with individually assigned flow rates
Drug deliveries in a multi-drug course may be separated by a time delay
Prefilled or pharmacy filled drug units 2 (or a mix of both) may be included
Different terminator units 3 may be used depending on the complexity of the delivery, the capabilities of the pharmacy, and the electronic health record needs Different drive units 1 may be provided to the patient depending on the complexity of the delivery
Regimen changes, such as change in volume or number of drugs, can occur.

A select few combinations of the above variables are described below in increasing complexity.
A single drug unit 2 is filled by the pharmacist. The pharmacist programs a level 1 terminator unit 3 using a physical tool with the parameters for the dispense. The pharmacist connects the drug unit 2 and terminator unit 3 and sends the assembled/pre-assembled device to the patient. The patient, previously provided with a low-power drive unit 1, attaches the received drug course and does the infusion.
Two drug units 2 are filled by the pharmacist. The pharmacist programs a level 2 terminator using the electronic health record integration with the parameters for the dispense. The pharmacist connects the drug units 2 and terminator unit 3 and sends the assembled/pre-assembled device to the patient. The patient, previously provided with a low-power drive unit 1, attaches the received drug course and does the infusion.
Three drug units 2 are filled by the pharmacist. The pharmacist programs a level 2 terminator unit 3 using the electronic health record integration with the parameters for the dispense. In particular, this course requires a delay between the delivery of drug 2 and drug 3. The pharmacist connects the drug units 2 and terminator unit 3 and sends the assembled/pre-assembled device to the patient. The patient, previously provided with a high-power drive unit 1, attaches the received drug course and does the infusion.
Four drug units 2 are filled by the pharmacist. The parameters for the dispense are recorded in the cloud interface for the product. In particular, this course includes a contingent medication to be delivered in event of an infusion reaction. The pharmacist connects the drug units 2 and sends the assembly to the patient. The patient, previously provided with a high-power drive unit 1 and a level 3 terminator unit 3 attaches the received drug course to those components. The terminator unit 3 connects to the cloud and downloads the information about the course before starting the infusion. If the device/system detects an infusion reaction it will stop delivery of the standard drugs and immediately delivery the contingent medication. If no reaction occurs the contingent medication remains unused.

As the assembly of the device is simple, the pharmacy may also provide the drug unit(s) and/or terminator unit 3 loose or pre-assembled to be put together by the patient.

Although liquid parenteral modules/units are shown in the illustrative examples above, the present invention need not be limited to liquid or parenteral routes, or even medication delivery at all. Modules assembled in the system may have other functionalities that are situational in nature and tailored to specific use cases for a drug delivery device, disease area treatment, or medication regimen. These modules may be advantageously provided as a separate module or, alternatively, the terminator unit 3 or combinations thereof to program various aspects of a controller, such as that described in U.S. Application No. 63/226,498. Some examples of modules that may be combined, by way of example and not limitation, include:
Interlock Modules: These allow contingent administration of one or more medications contained in a drug unit 2 based on electronic health record information or clinician input/intervention. The information could be, by way of example, laboratory values, physiologic values, or qualitative information (e.g., from a telemedicine or physical examination). This interlock functionality is used to ensure that patients may safely receive one or more medications administered by the system.
Clinical Trial (CT) Modules: Modules/units may be included specifically for the purposes of monitoring various aspects of a clinical trial or to collect information associated with a clinical trial. Aspects of a clinical trial could include one or more clinical trial test conditions for the medication being studied in the trial, clinical trial identifier, clinical trial medication, clinical trial test condition, clinical trial randomization schedule identifier, or patient identifier. A module may also be used to disguise or “blind” a clinical participant to one or more of the medication (or a placebo medication in a randomized clinical trial), clinical trial test condition, clinical trial randomization schedule, or clinical trial sponsor.
Infusion Reaction Modules: Modules/units may be included to allow detection of, or response to, systemic infusion reactions caused by administration of one or more medications contained within the drug delivery system. Such a module may be configured to detect suspected infusion reaction and direct the system to deliver (i.e., contingently) appropriate therapeutic treatment automatically or through intervention by a healthcare provider. The module may utilise historical data from a single patient, groups of patients, historical data from one or more prior clinical trials with the therapeutic medication being administered, or probabilistic estimates to determine whether an infusion reaction is occurring. The infusion reaction module may also be optionally configured to allow “rechallenge” protocols to be implemented, whereby prior medications associated with an infusion reaction are reintroduced to a patient at a slower administration rate, which is then progressively increased while monitoring for reaction. If desired, the IR functionality may be combined with interlock modules to provide additional levels of patient safety and data continuity for future medication administrations. Thus, IR modules may serve a diagnostic, treatment, or preventive roles, or combinations thereof, all based on the medication(s) being administered and patient characteristics.
Oral Medication Interlock/Sequencing: When administering certain therapeutic medications, prophylactic medicines may be administered to a patient before (pre-medication) or after a therapeutic medication (post-medication) to avoid systemic infusion reactions or ease discomfort from a therapeutic medication's side effects. The pre-medication and post-medications may also comprise part of a medication regimen or medication order set. Modules may be included to either contain and dispense oral pre and post medications as part of the sequence, and may be optionally interlocked to prevent administration of interposed medication(s) (e.g., a biologic) until the appropriate oral medications have been taken, and any requisite time delay has taken place, such as that contained in an electronic health record, order set, or medication regimen.
Radioisotope interlock: The system may be configured to prevent administration of a radionucleotide (e.g., radioligand therapy) until the system is properly configured. Alternatively, the system may be interlocked contingently, based on assembly of the proper shielded medication reservoir and/or tubing set to prevent inadvertent radiation exposure to a patient or healthcare provider. The module may optionally be interfaced to an electronic health record system, be configured to detect a relevant medication requiring such system, and activate the interlock functionality contingently.

In some embodiments, as those described earlier, the drive unit 1 can contain the controller (drug delivery controller 14) that is programmed by the terminator unit 3 (drive unit software is a generalised controller software and is programmed (i.e., customised) by the assembled system). Some or all units may be virtualised in software as opposed to physical embodiments.

To provide flexibility in manufacture and dispensing, the terminator unit 3 may contain a wide variety of communication capability. During manufacture, the core drive unit 1 and drug units 2 may be mass-manufactured for broad applicability and low cost, and different terminator unit 3 variants may be more easily manufactured (or changed as technology evolves as described previously). During dispensing, the drug units 2 are filled or assembled as described previously, and the appropriate terminator unit 3 variants may be selected based on the use case, region, patient needs, or other relevant factors.

There are several use cases that can be contemplated as illustrative examples, and not limitation:
Supporting software in pharmacy creates a bridge between existing EHR software and the device hardware to enable simple programming of course parameters
Electrical interface allows error-proofing features such as genuine part detection, tamper detection, incorrect sequence detection
Pharmacy is still responsible for course correctness. Patient interaction is error proofed by not providing the patient the ability to modify dispense parameters.
When the patient connects a preassembled series of drug modules 2 and terminator unit 3 to their drive unit 1 the custom course information is communicated to the drive unit 1 via the common interface (i.e. interfaces 11, 21, 31).

The terminator unit 3 does not necessarily need to be the terminating component at the end of the system. In some scenarios, the drive unit 1 may also serve as the terminator. If a drive unit 1 were designed to be cloud-connected from the beginning, a separate terminator component would not be needed. Course information would reach the drive unit 1 at the patient home via the cloud. If a drive unit 1 were designed to be disposable it could serve a dual function as a terminator unit 3. It would be programmed in the pharmacy before shipment using the same mechanism.

If drug units 2 are individually programmed with drug parameters, the last drug unit in the system may be identified as such. It could communicate that information to the drive unit 1 to acknowledge that a completed drug course was provided to the patient.

In this case, a simple passive terminator unit 3 may be used to cover exposed connections on the last drug module. Alternatively, the connections may be self-terminating inherently.

The present invention creates a medicament delivery system as well as an electronic programming and control system for a drug delivery device. Also, a method of assembly of the system is provided. It allows modularity and programmability for customized drug courses, integrates easily into the lives of HCPs and patients, provides the needed safety interlocks for a variety of therapeutic medications and medication regimens, and is independent of the drive technology. Moreover, decoupling the drug delivery components from the controller offers several advantages:
Sustainability: Separating the drive, reservoirs, and programming functions allows relatively expensive drive components to be reusable. Different locomotives could be used depending on the volume of drug delivery (for pump and battery cost savings).
Future proofing: The same sequence of drug units could be used with multiple different drive units and/or terminator units. This removes the need to put connectivity in the drive unit itself. The drives or termination units may be entirely different designs, or different generations of a single design. This allows flexibility to tailor the system to specific disease areas, regions, and cost targets; also, to adapt as technology changes without requiring changes to the drug units
Number of medications: Any number of drug units can connect in any order by a pharmacist. Drug units can be generic or pre-filled with set doses of particular drugs.
Changing regimens: Regimens can change over a course of therapy for an individual patient. For instance, a single medication (e.g., a biologic medication) may have an initial series of more frequent loading doses followed by less frequent maintenance doses. A single medication may also feature an up-or down-titration scheme whereby doses at each dosing interval are increased or decreased on a predetermined schedule. A medication regimen may feature one set of medications at a first administration timepoint, and additional (or different) medications at a second administration endpoint. In all these instances, the present invention allows the medications to be simply assembled into the correct sequence and administered with the same drive unit, adapting delivery to support a particular course, to allow complex treatment regimens, or to respond to standard-of-care advancements.

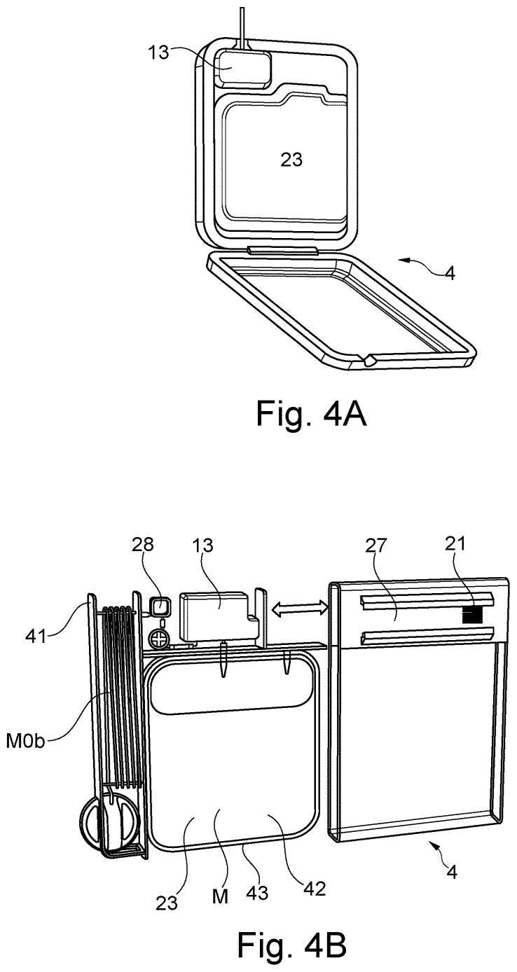

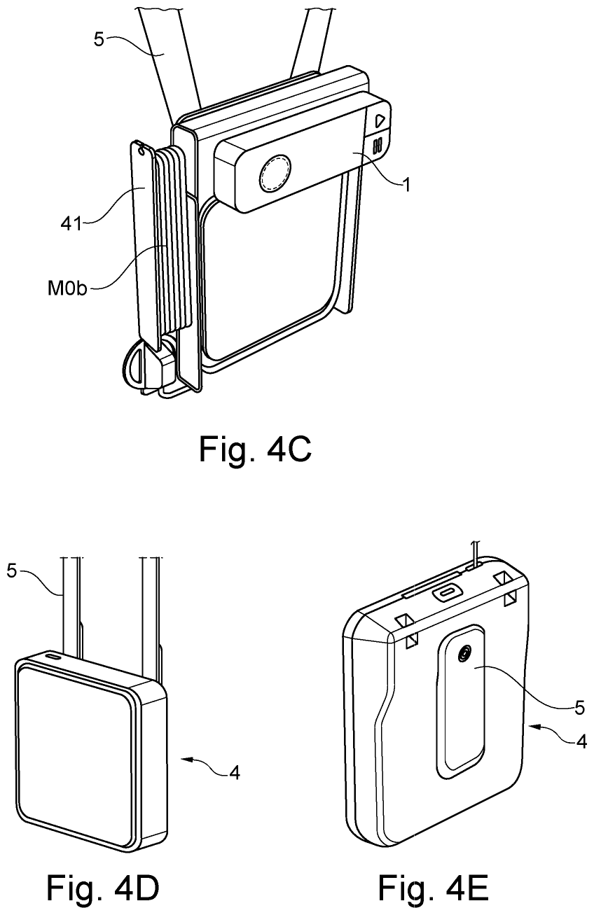

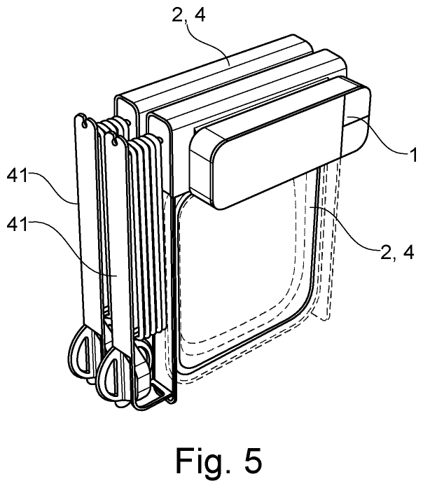

The present disclosure further relates to a drug delivery system containing a single module of medication as shown in FIGS. 4A and 4B.

The drug delivery system shown in FIG. 4A comprises a reusable outer shell 4 (body) containing a single module of medication in a drug reservoir 23.

The shown medicament delivery device comprises a reusable body 4 comprising a pump assembly such as a fluid pressure power source 13. The fluid pressure power source 13 can be a pneumatic power source or a hydraulic power source. The pneumatic power source is configured to output gas to the cassette for expelling contained medicament by increasing the fluid pressure of the gas, and the hydraulic power source is configured to output liquid to the cassette for expelling contained medicament by increasing the fluid pressure of the liquid. The fluid pressure power 13 source may be viewed as a drive unit 1 and may comprise, e.g., a power source 15 and/or a drug delivery controller 14. In particular, the medicament delivery system shown in FIG. 4A may be formed as one device rather than at least one drug unit 2 and a separably provided drive unit 1. In a preferred example, the medicament delivery device is a portable device.

The fluid pressure power source 13 is configured to generate/release pressurized fluid, e.g., liquid or gas, and deliver the pressurized fluid into the shell 4 forming a fluid-tight chamber. In a preferred example, the fluid pressure power source 13 is configured to generate/release pressurized gas. In one example, the fluid pressure power source 13 can be a pressurized gas canister. In a preferred example, the fluid pressure power source comprises a piezo pump. Alternatively, the fluid pressure power source 13 comprises a motor-based fluid pump, e.g., a diaphragm pump or a piston pump.

In this example, the reusable body 4 (shell 4) comprises a rigid container chamber where the flexible portion of the medicament container and the fluid-tight chamber is at least partially located within the rigid container chamber of the container carrier. The drug reservoir 23 may be formed as a flexible medicament bag 23. A flexible tube Mob may be attached to the flexible bag/drug reservoir 23. Alternatively, a flexible tube Mob may be attachable, e.g., via a valve such as a pinch valve, to the flexible bag/drug reservoir 23.

In one example, the fluid pressure power source 13 is attached to the inner section of the reusable body 4, as shown in FIG. 4A.

In FIG. 4A, the drive unit 1 and the drug unit 2 are both formed as a single device, i.e., incorporated in the shell or reusable body 4. In other words, in the example of FIG. 4A, the drive unit 1 may be integrally formed with the drug unit 2. Thus, a terminator unit 3 as described above may not be necessary.

In another example, the fluid pressure power source 13 is attached to a cassette and the cassette is attached to the inner section of the reusable body 4, as shown in FIG. 4B. The cassette may in the following denote the part that is inserted into the reusable body 4 as described below. The direction of insertion and removal is indicated by an arrow in FIG. 4B.

In one example, the reusable body 4 comprises two covers connected together, e.g., via a hinge, as shown in FIG. 4A. Alternatively, the reusable body 4 comprises a wall around the inner section, in other words, the inner section of the reusable body 4 is the space with a boundary that is provided by the wall, as shown in FIG. 4B. In this example, the wall is formed around an opening. The cassette is configured to be placed into the inner section through the opening, e.g., the user inserts the cassette into the inner section of the reusable body 4 through the opening. In this example, the cassette can be attached to the inner section of the reusable body 4 via a groove-and-ridge connection but other means are also feasible.

It should be noted that in the example as shown in FIG. 4B, instead of being attached to the cassette, the fluid pressure power source 13 can be releasably attached to the inner section of the reusable body 4 independently. For example, the fluid pressure power source 13 can be inserted into the inner section of the reusable body 4 followed by the cassette being inserted into the inner section. In this example, the fluid pressure power 13 source can be reusable or disposable. Furthermore, in one example where the medicament container M is a combination of a flexible bag 23 and a delivery tube Mob extending from the flexible bag 23, as shown in FIG. 4B, the flexible bag 23 of the medicament container is placed within a fluid-tight chamber 42 of the cassette. The delivery tube Mob may be wound around a frame 41 of the cassette. In this example, the cassette is configured to be partially placed within the inner section of the reusable body 4, as shown in FIG. 4C. When the cassette is placed into the reusable body 4, the frame 41 and the delivery tube Mob may be positioned outside of the inner section of the reusable body 4. Additionally, in another example, the cassette may be provided with a filter 28 connected to the delivery tube Mob. In a preferred example, the filter 28 is configured to expel air bubbles that are contained in the contained medicament such that the air bubbles will not be delivered into the user's body.

Furthermore, instead of having the fluid-tight chamber 42 as a part of the cassette as mentioned above, the fluid-tight chamber 42 can be formed by the cassette and the reusable body 4. For example, the cassette may comprise a medicament container frame 43, as shown in FIG. 4B, configured to surround the medicament container 23. In this example, the medicament container 23 is attached to the medicament container frame 43 and is configured to be inserted into the inner section of the reusable body. When the medicament container frame 43 is placed into the inner section of the reusable body 4, the inner section of the reusable body 4 together with the medicament container frame 43 form the fluid-tight chamber. In this example, the cassette can be made of less plastic material, thus, the cost of the cassette can be reduced. In another example, the medicament container frame 43 comprises a connector to the fluid pressure power source 13, for example, a valve. Therefore, the fluid pressure power 13 source can only be activated to release the fluid into the fluid-tight chamber once the medicament container frame 43 is inserted into the inner section of the reusable body 4. Additionally, the reusable body 4 may comprise a connection track 27. In one example as shown in FIG. 4B, the connection track 27 is formed by two ribs. The connection track 27 enables multiple reusable bodies 4 to be attached together. For example, the reusable body 4 may comprise the connection track 27 at one side and a connection protrusion at the other side opposite to the connection track 27. The connection protrusion is configured to be attached to the connection track 27 by being slid along the connection track 27. In this example, the reusable body may comprise a communication spot 21 (communication interface/drug unit interface) configured to connect to a counter communication unit of another reusable body. For example, the communication spot 21 is arranged within the connection track 27, as shown in FIG. 4B. The counter communication unit is arranged in the connection protrusion. The communication spot 21 can be a conductive spot configured to be in contact with and electrically connected to the counter communication unit. Alternatively, the communication spot 21 can be an RFID/NFC circuit that is configured to be connected with the counter communication unit via a contact free connection.

When the drug reservoir 23 and the connected features described above, which together may also be referred to as a cassette, are inserted into the reusable body 4 (cf. FIGS. 4B and 4C), the reusable body 4 with the cassette placed therein may also be referred to as a drug unit 2.

As shown in FIG. 4C, a drive unit 1 may be attached to the outer shell 4 forming the drug unit 2 and may control medicament delivery as described above. The drive unit 1 may also be referred to as a user interface. The drive unit 1 may thus control operation of the device by user input and provide visual and/or tactile and/or auditive information to a patient. An additional terminator unit 3 as described above may not be necessary

Moreover, in FIGS. 4C and 4D, the drive unit 1 may not comprise the pump assembly 13, and consequently which may be provided in the shell 4 (reusable body). As described above, the drive unit 1 according to FIGS. 4C and 4D may comprise at least one of a drive unit interface 11, a drug delivery controller 14 or a power source 15. The drive unit interface may correspond to and be in communication with the communication interface 21 of the reusable body 4 (drug unit 2), wherein the communication may be by contact, wired, or wireless.

The drug delivery system may further comprise a user wearable feature 5 such as a belt, or a strap as shown in FIGS. 4C and 4D. Alternatively or in addition, the user wearable feature 5 may be a belt clip, as shown in FIG. 4E.

The medicament delivery system according to FIGS. 4A to 4E may thus be belt worn, placed to the side of a patient, or worn over shoulder with a strap. The system is well-suited for participants who prefer unitary weight distribution, a smaller and lighter concept, or the device weight largely supported by the torso. A looser device fit can accommodate more bodies and wear configurations. The patient is unburdened from infrastructure for additional reservoirs and freed from carrying an entire regimen (e.g., multiple medications in separate flexible bags) at once.

The reusable outer shell 4 may contain a battery (power source 15), drive unit 1, and reservoir interface 22. This can be integral, as in shown in FIG. 4A, or may be optionally detachable or separable, as shown in FIG. 4B. Pumps in the fluid path (e.g., Weibel pumps) may be incorporated into the reservoir. Mechanical or pneumatic interface points may be included based on the drive system selected. For a pressure drive system or a peristaltic pump, the pump 13 may be incorporated into the reusable portion of the system (e.g., the shell 4), as the pump is not in the fluid path. For a pump driven system that is in the fluid path (e.g., Weibel pump, split cam), the pump 13 may be included in the disposable component (medicament bag 23).

The design is intentionally insensitive to orientation or gravity. A single infusion line may exit the drug reservoir 23 and can be used with either thigh or abdomen sites. Tubing management, e.g., a frame 41, may be optionally included, as may be the infusion needle, as show in FIG. 4B. Alternatively, the tubing may be terminated in a modular connector, and the tubing set and needle(s) may be attached separately as part of the use process.

The system according to FIGS. 4A to 4E has a simple set of use steps that may be performed multiple times. The patient inserts the drug cassette into the injector as seen in FIG. 4B (arrows), attaches the needles to the injection site and simply presses a start button to administer the full treatment regimen. For multiple medication regimens, the device may remind the user when to proceed, or reject medications loaded out of sequence.

A removable strap 5 and/or belt clip 5 allow for various wear configurations, as seen in FIGS. 4C-4E. A fabric panel in the shell 4 can be easily customized to suit different pharmaceutical brands. If provided, the hinged outer cover 4 may be designed to be reminiscent of consumer products. A soft closure using magnets may be provided and may have a fabric cover that may be easily customized for different pharmaceutical brands.

FIG. 5 shows a further embodiment related to FIGS. 1 to 3 as well as 4B and 4C. In this embodiment, two reusable bodies 4 with two cassettes inserted respectively (see above) are attached to one another. The drug units 2 respectively formed by a reusable body 4 and a cassette may be attached via the connection track 27 on one drug unit 2 and a corresponding connection feature (not shown) on the other drug unit 2, wherein the corresponding connection feature is provided on a side opposite to the connection track 27. Also, more than two drug units 2 may be attached to each other via the connection described above.

The drive unit 1 (user interface) is attached to one of the two reusable bodies 4. The drive unit 1 may also be connected via the connection track 27 and a corresponding connection feature (not shown). In this embodiment, the drive unit 1 is configured to detect how many cassettes, i.e., shells/drug units 2, 4, are connected, via the connection between the communication spot 21 and the drive unit interface 21. The drug units 2 may also be in communication among each other via the respective drug unit interfaces 21 as described above.

The drive unit 1 may thus control the medicaments in different cassettes 2, 4 to be delivered accordingly.

As shown in FIGS. 1 and 2, one or more drug reservoirs 23 are stored in respective (rigid) cartridges (drug units 2) and filled by the pharmacist to form one or more drug units 2. Then, they may be securely assembled in sequence (e.g., using permanent snaps or dual side tape) and capped with a disposable interface (e.g., drive unit 1 as described above). Dedicated electrical, optical, pneumatic, or fluidic connectors may be provided on each cartridge to communicate with the drive unit 1 (i.e., drive unit interface 11, drug interface 12, drug unit interface 21, drug interface 22).

For medicament delivery, the patient receives the pre-assembled drug units 2 and installs them into the reusable base (drive unit 1). For example, a push button attachment mechanism may secure the complete stack of drug unit(s) 2 and drive unit 1 (and terminator unit 3, if applied) and allows for easy disassembly when done. The base 1 may be provided with a dedicated pneumatic connection for when an emergency drug is required. The patient interface may always be on top regardless of the number of drug units 2 used.

Alternatively, as shown in FIG. 5, the drug reservoirs 23 may be removable from the outer shell 4, and may be inserted by pharmacy and stacked in a variety of configurations, also terminated with a disposable interface (drive unit 1). This allows for a small form factor if desired by allowing stacked or unitary operation (one module at a time, swapped manually). The above configurations may be provided with an integrally connected tubing set, needle, and optional tubing management features.

FIG. 5 shows a configuration with two drug units 2 (shells 4/cassettes), but as described above, only one or more than two drug units 2 may also be connected in the manner outlined in this specification.

In case a plurality of drug units 2 are used, the belt/strap/clip 5 may be provided only on the drug unit located nearest to a patient's body. The belt/strap/clip 5 may be provided on a side of the drug unit 2 opposite of the connection track 27.

In one or more embodiments of the present disclosure, the belt, strap, or clip 5 may be antimicrobial, antifungal, or antiviral. In particular, one or more fabric items, such as belt 5 (e.g., around a waist of a patient), shoulder straps 5, or harness 5, or portions thereof, may be provided with one or more of a persistently antimicrobial, antifungal, or antiviral agent.

Such a coating may include fibers woven into the fabric material (e.g., silver fibers), may be provided through a secondary coating, spray, or dipping operation, or by selecting an outer fabric layer featuring persistently antimicrobial, antifungal, or antiviral properties. For instance, the coating could comprise NordShield (Nordic BioTech Group Ltd.), as described in US publication number US 2021/0204552 A1 to Holopainen, or a fiber could be selected containing a similar compound, as detailed in US 2019/0271111 A1, also to Holopainen.

In one or more embodiments of the present disclosure, one or more rigid aspects of the designs, such as outer housings, connection points, or interfacing surfaces for the medication dispensing fluidics are provided with (i.e., molded in, molded with) a compound featuring persistently antimicrobial, antifungal, or antiviral properties. Alternatively, a compound featuring persistently antimicrobial, antifungal, or antiviral properties may be applied to the molded (i.e., finished) components through secondary processes (e.g., chemical vapor deposition), spraying, or dipping processes. This may apply to an outer housing of at least one of the drive unit 1, the drug unit 2, the terminator unit 3, or the shell 4. In FIG. 4B, particularly the area of the connection track 27 and/or the shell 4 receiving the cassette may be antimicrobially, antifungally, or antivirally provided.

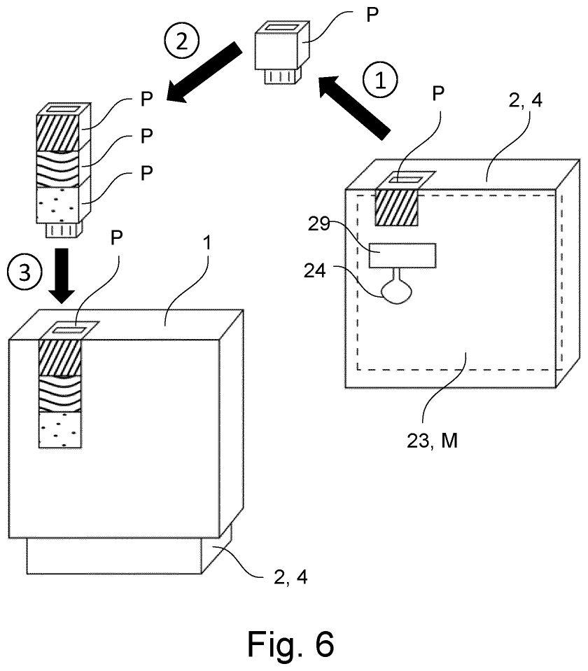

FIG. 6 is a depiction of an embodiment of the present disclosure compatible with the above described embodiments. In particular, FIG. 6 shows a stackable control system useable with, e.g., the drug unit 2 stacks of FIGS. 1, 2 and 5.

At least one drug unit 2 (cassette) is provided. The drug unit 2 may contain a drug reservoir such as a flexible medicament bag 23 or may comprise a shell 4 having a fluid-tight chamber 42 as described above. Optionally, the drug unit 2 may comprise drug unit electronics 29, e.g., a PCB (printed circuit board) comprising electronics for controlling medicament delivery and/or a drug unit identification 24 which may be an RFID chip, NFC chip or other passive or active electronics allowing identification of the drug unit 2 and the contained drug, prescription details, or administration parameters related to the contained drug.

Furthermore, the embodiment comprises a drive unit 1 configured to house at least one drug unit 2.

In this embodiment, the drug unit 2 further comprises a removable programming key P. The programming key P may comprise a male connection and a female connection allowing it to be connected to other programming keys P as shown in FIG. 6. The programming key P may be formed as a kind of USB component. However, other forms of providing contact between programming keys P allowing an exchange of information may also be feasible.

The programming key P may comprise logic and memory, i.e., may be provided with internal circuitry and supporting electronics. The programming key P and its connectors may further function as a drug unit interface 21 providing communication between the drug unit 2 and the drive unit 1 as well as different drug units 2.

As shown in step 1 of FIG. 6, a portion of the medication cassette is the removable programming key P corresponding to a respective drug unit 2.

As shown in step 2 of FIG. 6, a plurality of programming keys P may be connected or “stacked” via the cooperating connectors. This may be similar to how USB connections are be made. As the programming keys P are assembled, a “train” (i.e., a stack) is built without mechanically connecting the drug unit 2 (cassettes). Optionally, although not shown here, there may be a locking mechanism provided to prevent separation or re-ordering of the assembly once constructed, or as it is constructed, or “stacked”.

No termination unit may be required as the programming keys P are configured to self-terminate.

As shown in step 3 of FIG. 6, the stack of programming keys P forms an assembly, with the male connector used to make electronic communication with the drive unit 1. In the drive unit 1, at least one drug unit 2 may be connected. Also, a plurality of drug units 2 may be sequentially connected to the drive unit 1 according to a prescription. The different programming keys P belonging to different drug units 2 are indicated by different patterns in FIG. 6. That is, the programming keys P may be coded, e.g., colour-coded, having a barcode, a QR code or text/images for distinguishing the programming keys P. This programming key P assembly may be constructed by a pharmacy, dispensed to the patient (or nurse) by the pharmacy, and inserted by the patient or nurse into the reusable drive unit 1.

In an embodiment, a shift register may be used to describe the movement of information from the drug unit 2 and terminator units 3 to the drive unit 1. In this case, the programming keys P, as they are stacked, together make up a shift register. When inserted into the drive unit 1, the stacked programming key P assembly pass course information from key P to key P. When it reaches the drive unit 1, it is stored in memory for use during the drug dispense. The shift register architecture is serial, meaning the physical order of the drug units 2 is preserved in the order in which information arrives at the drive unit 1. Each additional programming key P includes its relevant information in the broader shift register, so the drive unit 1 has an awareness of exactly what is connected to it and in which order. Shift register-style serial communication may be implemented with one of several standard communication protocols (SPI, UART) or via a custom protocol.

In a preferred embodiment, removal of the programming key P leaves no active electronics in the disposable drug unit 2 (cassette), as this simplifies disposal and improves sustainability of the used (i.e., empty) drug unit 2.

As outlined above and alternatively, the drug unit 2 (cassette) may be provided with supporting drug unit electronics 29 on a PCB, and/or with an identification 24 such as an RFID/NFC antenna. Such a system may be used for drug unit 2 interrogation by the drive unit 1 when inserted, providing a cross check between the cassette RFID/NFC and the stacked programming key P in the drive unit 1. Such a system may also be used for disposal of the empty cassette.

As outlined above, a plurality of drug units 2 may be connected at once to the drive unit 1 or sequentially according to a prescribed or desired regimen. Thus, the drive unit 1 may comprise an indicator (such as at least one LED or display) to communicate to the patient, which drug unit 2 is to be attached.

Moreover, in a drug delivery system according to the embodiments described above, communicating regimen parameters to a generic, reusable, multi-drug delivery device at the point of administration may be important. The parameters to be communicated to the delivery device may, e.g., be defined as follows:
Regimen Length—the total number of drug unit 2 (reservoirs) to be given back-to-back (e.g., three drug units 2)
Sequence or Order—the ordinal position of each drug unit 2 in the regimen (e.g., 1^{st}, 2^{nd}, 3^{rd})
Medication-specific Parameters—optionally, some parameters about one or more medications in the regimen, including but not limited to molecule name or delivery flow rate

Some existing devices (e.g., EMED ScIG, Koru Medical Freedom Edge) utilise user-sequenced workflows to deliver multiple medications. That is, a user attaches a first medication reservoir (e.g. syringe) to the drive mechanism, connects any necessary tubing components, inserts the required needles into the tissue, and begins dispensing that reservoir. When the first reservoir is complete, the user detaches the empty reservoir from the drive mechanism, and partially or fully removes the tubing components and needles. Then, the user attaches a second medication reservoir to the drive and completes tubing and needle assembly again.

In developing a large volume subcutaneous infusion device, the present disclosure is concerned with both user-sequenced and device-sequenced workflows. In device-sequenced workflow concepts, the user attaches all drug units 2 (reservoirs) to the drive unit 1 at one time and the device sequentially administers each reservoir.

The stack of drug units 2 described in the above disclosure relies on a device-sequenced architecture. If the drug units 2 are not in connection with one another and the drive unit 1, the regimen termination and length cannot be known by the drive unit 1 through the previously described system and methods. Additionally, a user-sequenced workflow cannot rely on the pre-assembled stack of drug units 2 to deliver in the correct order. Instead, the user must correctly choose and insert a specific drug unit 2 at multiple points throughout the regimen.

Therefore, it is desirable to explore alternative methods of communicating the parameters of regimen length, sequence, and optional medication-specific parameters to a generic user-sequenced drive unit 1.

This object may be met with the embodiment described below.

### Communicating Sequence and Medication-specific Parameters

Each drug unit 2 (medication cassette) may be provisioned with one or more wireless communication methods for communication with the drive unit 1 as described above. In an embodiment, the communication method is a passive RFID circuit (e.g., the identification 24 or the drug unit interface 21) located on an external surface of the drug unit 2, though other communication methods are possible. One or more RFID circuits (i.e., antennas and passive components) may be optionally included, each also optionally operating at the same frequency, or one or more discrete frequencies. For instance, a first frequency may be used for dispense information, such as flowrate, while a second frequency may be used to communicate sequence-related information. That is, the identification 24 may carry information on the drug. It is preferrable for the drug unit 2 of the communication method to be low-power or passive, as the drug units 2 may be single-use and disposable. In contrast, the communication functions on the drive unit 1 may be more power-intensive as this device has a longer life-span, adequate power supply for the drive mechanism, and may be provisioned for re-charging. The wireless communication module may be attached to the drug unit 2 in manufacturing or in pharmacy dispensing.

In an embodiment, the wireless communication module of the drug unit 2 encodes a position in the ordinal regimen sequence (e.g., first drug unit 2, second drug unit 2, etc.). In some embodiments, one or more drug units 2 in the regimen additionally encode(s) medication-specific parameters in this way. The presence and contents of medication-specific parameters need not be the same on each drug unit 2 in the regimen. When a drug unit 2 is loaded into the drive unit 1 for administration, the drive unit 1 may read this sequence information.

In an embodiment, prior to beginning administration, the user causes the drive unit 1 to sequentially communicate with each drug unit 2 in their regimen so that the drive unit 1 may read the sequence encoded therein. In some embodiments, this is done by bringing each drug unit 2 near to the drive unit 1 (e.g., waving the drive unit 1 over each drug unit 2). In other embodiments, this is done by temporarily inserting each drug unit 2 into the drive unit 1. As the drive unit 1 reads each drug unit 2, it stores the sequence information and begins to build a “virtual stack” of regimen information (e.g., a first medication, followed by a second medication, followed by a third).

One advantage using the wireless communication to build the virtual stack of drug units 2, is that the user may scan the drug units 2 in a different order than the regimen administration sequence without communicating incorrect sequence information to the drive unit 1. That is, for example, if a user first scans a drug unit 2 with sequence “third”, the existence of a “first” and “second” drug unit 2 is implied. At that time, the drive unit 1 cannot know the contents of these first two drug units 2, but may build a stack with “gaps” (i.e., ______, ______, followed by a third medication). It may be additionally possible to detect that a user has scanned a drug unit 2 in duplicate (e.g., two “first” drug units 2) and communicate a warning or error state.

### Communicating Regimen Length

Alternative to the use of a terminator unit 3, it is possible to terminate the building of the virtual stack or drug units 2 (and therefore define regimen length) through direct user input.

In one embodiment, the drive unit 1 has a simple push-button that is used to signal the end of “scanning” or “stack building” mode, allowing the user to transition to medication administration.

In an embodiment, a more advanced device interface to collect this input may be provided on the drive unit 1. For example, a “second” drug unit 2 (cassette) is temporarily inserted into the drive unit 1 to be read. After the drug unit 2 is removed, an additional decision point is presented to the user after each drug unit 2 is administered and removed: is this the end of the regimen or are there more drug unit 2? A button representing “end” with appropriate symbols or words may be provided on the drive unit 1 and may be illuminated. A second button representing “more cassettes” with appropriate symbols or words may be provided on the drive unit 1 and may be illuminated. In case of illumination, the buttons may be illuminated in different colours, by blinking or alternately illuminated to indicate the user to make a selection.

### Applications for Improved User Feedback

In an embodiment, the drive unit 1 may comprise a display configured to display the virtual stack of drug units 2 as it is being built in “scanning mode”. This may exemplarily be communicated through a segmented LED bar or series of symbols. As a “first” drug unit 2 is scanned, the first segment or symbol is illuminated, and so on. In this way, it is also possible to display “gaps” in the virtual stack of drug units 2 to the user as described above. For example, if a user first scans a “third” drug unit 2, only the third segment or symbol will illuminate with the first and second segments staying dark. Alternatively, the third segment may illuminate and the first and second segment may illuminate differently (e.g., with a different colour light, brightness, or flashing pattern) to prompt the user to keep scanning.

More detailed user interfaces on the drive unit 1 are possible including the use of LCD screens displaying the medication-specific parameters of each medication in the stack. In an embodiment, the drive unit 1 may additionally wirelessly communicate with a mobile device (e.g., smartphone, tablet) to provide the user with more detailed information about the virtual stack of drug units 2 than is provided on a user interface (UI) of the drive unit 1.

When the drive unit 1 is transitioned into “administration mode”, the information contained in the virtual stack of drug units 2 may continue to provide improved user feedback.

In an embodiment, the drive unit 1 may instruct the user as to which drug unit 2 to insert next by referencing the position in the virtual stack of drug units 2. This instruction may be provided on the drive unit 1 or mobile device and may be provided at varying levels of detail through text or symbols. A few non-conclusive examples are provided below.
The medication name—“insert pembrolizumab”
The ordinal position of the drug unit 2/cassette—“insert the third cassette”, illuminating a numeral 3, illuminating a third segment
The colour of the drug unit 2—“insert the blue cassette”, illuminating a blue symbol

When a drug unit 2 is inserted into the drive unit 1, a drive unit processor (e.g., the drug delivery controller 14) may additionally re-read the encoded information. By referencing the newly read encoded information against the expected information based on the current position in the virtual stack of drug units 2, it is possible to verify whether or not the user has inserted the correct drug unit 2. If the user has not inserted the correct drug unit 2, it is further possible to provide a warning or error state to the user.

Other aspects, features, and advantages will be apparent from the summary above, as well as from the description that follows, including the figures and the claims.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below.

Furthermore, in the claims the word “comprising” does not exclude other elements or steps, and the indefinite article “a” or “an” does not exclude a plurality. A single unit may fulfil the functions of several features recited in the claims. The terms “essentially”, “about”, “approximately” and the like in connection with an attribute or a value particularly also define exactly the attribute or exactly the value, respectively. Any reference signs in the claims should not be construed as limiting the scope.

Some aspects of the invention are summarised as follows.
1. Drug delivery system, the system comprising:
   a drive unit,
   wherein the drive unit comprises a drive unit interface, a drug interface, a pump assembly or a motor assembly configured to deliver a drug, a drug delivery controller and a power source;
   at least one drug unit connectable to the drive unit,
   wherein the drug unit comprises a drug reservoir for storing a drug, an identification comprising information on the drug, a drug interface communicated with the drug reservoir, and a drug unit interface,
   wherein the drug unit interface is configured to communicate with the drive unit interface,
   wherein the drug interface of the drug unit is configured to communicate with the drive interface of the drive unit; and
   a terminator unit connectable to the drug unit,
   wherein the terminator unit comprises one or more components of a system controller, a terminator unit interface configured to communicate with the drug unit interface, and
   wherein the terminator unit is configured to close at least one of the drug unit interface and the drug interface of the drug unit.

2. Drug delivery system according to clause 1, wherein the drive unit interface and the drug unit interface comprise at least one of a mechanical or electrical interface.
3. Drug delivery system according to clause 1 or 2, wherein the drug interface of the drive unit and the drug interface of the drug unit comprise at least one of a mechanical connection, a sealed pneumatic connection, or an electrical connection.
4. Drug delivery system according to one of the preceding clauses, wherein the system comprises a plurality of drug units,
   wherein each of the plurality of drug units comprises identical drug interfaces and drug unit interfaces,
   wherein the drug units are configured to be connected via the drug interface and the drug unit interface,
   wherein the drug units are configured to be connected between the drive unit and the terminator unit.
5. Drug delivery system according to clause 4, wherein the drug units are configured to be connected between the drive unit and the terminator unit to form a stack, and/or
   wherein the drug units are configured to be connected between the drive unit and the terminator unit in an order corresponding to a planned order of administration to a patient.
6. Drug delivery system according to one of the preceding clauses, wherein the units are configured to communicate via a wired or wireless connection, preferably via the electrical interfaces.
7. Drug delivery system according to one of the preceding clauses, wherein the drug delivery controller is configured to control the pump assembly or motor assembly for delivering the drug to a patient, and
   wherein the drug delivery controller preferably comprises a non-volatile memory.
8. Drug delivery system according to one of the preceding clauses, wherein at least one of the drive unit, the drug unit or the terminator unit comprises at least one user interface element configured to control, indicate, or control and indicate a system status.
9. Drug delivery system according to clause 8,
   wherein the at least one interface element is at least one of a button, LED or screen.
10 Drug delivery system according to clause 8 or 9,
   wherein the system status indicates at least one of a correct connection of the units, sequence of the units, information on expiry of the drug, battery level, dispense parameter information, dispense error information, dispense readiness information, dispense status information, connection status, patient clinical characteristics, occlusion status of a tubing set, or needle used with the system.
11. Drug delivery system according to one of the preceding clauses, wherein at least one of the drug unit or the terminator unit comprises a wireless or wired connection module configured to communicate with at least one of a server, the internet or a terminal.
12. Drug delivery system according to clause 11,
   wherein the wireless or wired connection module is configured to receive and transmit drug delivery information,
   wherein preferably, the drug delivery information is received or transmitted during filling of the drug reservoir within the drug unit, and/or
   wherein preferably, the drug delivery information corresponds to one or more aspects contained in an electronic health record system for a patient using the drug delivery system.
13. Drug delivery system according to one of the preceding clauses, wherein the drug unit further comprises a temperature sensor configured to sense at least one of a temperature of a drug stored in the drug reservoir or an ambient temperature where the drug delivery system is used.
14. Drug delivery system according to one of the preceding clauses, wherein the identification is stored in an electronic memory, an RFID tag, a 2D barcode, a 3D barcode, a data matrix, or a QR code.
15. Drug delivery system according to clause 14,
   wherein the identification comprises information on at least one of a medication contained a drug unit, a volume of the drug reservoir, volume of medication within the drug reservoir, drug delivery information, dispense parameters of the drug, flow rate of the drug, drug lot number, patient identifier, an identifier of a medication in a sequence of one or more medications, expiration date, a temperature condition of the drug, administration date, administration time, medication cycle number, serialization/identifier information for one or more of the medication, or drug unit.
16. Drug delivery system according to clause 15, wherein the system controller is configured to read the identification and communicate with the drug delivery controller to perform drug delivery according to the identification.
17. Drug delivery system according to one of the preceding clauses, wherein one or more components of the system controller is reconfigurable and/or programable.
18. Drug delivery system according to one of the preceding clauses, wherein the terminator unit is reusable or disposable.
19. Method for assembling a system according to one of the preceding clauses, the method comprising:
   filling at least one drug into the drug reservoir of at least one drug unit prior to assembly of the system,
   assembling the drug delivery system by connecting the drive unit, at least one drug unit and the terminator unit, and
   reading, by the system controller, the identification of the at least one drug unit.
20. Drug unit for use in a system according to any one of clauses 1 to 18,
   wherein the drug unit comprises a drug reservoir for storing a drug, an identification comprising information on the drug, a drug interface communicated with the drug reservoir, and a drug unit interface, and
   wherein the drug unit is configured to be connectable to a drive unit and a terminator unit.
21. Drug delivery system, the system comprising:
   a pump assembly configured to deliver a drug;
   at least one drug unit,
   wherein the drug unit comprises a drug reservoir for storing a drug and being connectable to the pump assembly, and at least one shell configured to receive the pump assembly and the drug reservoir.
22. Drug delivery system according to clause 21, wherein the drug unit comprises two shells connected by a hinge or snap fit, wherein the pump assembly and the drug reservoir are provided between the shells.
23. Drug delivery system according to clause 21, wherein the drug unit comprises a medicament container frame and the drug reservoir and the pump assembly are provided on the medicament container frame,
   wherein the medicament container frame is received in the shell.
24. Drug delivery system according to clause 23, wherein the drug unit further comprises a filter configured to filter a drug.
25. Drug delivery system according to clause 23 or 24, wherein the drug unit further comprises a tube frame configured to receive a flexible tube.
26. Drug delivery system according to any one of clauses 23 to 25, wherein the drug unit comprises a connection track and/or a connection feature, wherein the connection track and/or connection feature is/are configured to attach drug units to each other.
27. Drug delivery system according to clause 26, wherein the connection track and/or connection feature is/are provided on the shell.
28. Drug delivery system according to any one of clauses 23 to 27, wherein the drug unit comprises a communication interface, wherein the communication interface is provided on the shell and/or wherein the drug unit comprises an identification, said identification being provided on the shell.
29. Drug delivery system according to any one of clauses 21 to 28, further comprising a drive unit,
   wherein the drive unit comprises a drug delivery controller and a power source.
30. Drug delivery system according to clause 29, wherein the drive unit comprises the pump assembly, and
   wherein the drive unit is provided in the at least one shell.
31. Drug delivery system according to clause 29, wherein the drive unit comprises a connection feature and a communication interface,
   wherein the drive unit is configured to be separably attached to a connection track of the drug unit, and
   wherein the communication interface is configured to communicate with a communication interface of the attached drug unit.
32. Drug delivery system according to any one of clauses 21 to 31, wherein the drug unit comprises a belt and/or a strap and/or a clip for attaching the drug unit to a patient.
33. Drug delivery system according to clause 32, wherein the belt and/or strap and/or clip is coated with a material having antimicrobial and/or antifungal and/or antiviral properties, or
   wherein the belt and/or strap and/or clip is at least partially formed of a material comprising antimicrobial and/or antifungal and/or antiviral properties.
34. Drug delivery system according to any one of clauses 1 to 18 or 21 to 33, wherein the drug unit comprises a removable programming key,
   wherein at least one drug unit is connected to the drive unit, and
   wherein the removable programming key is connected to the drive unit.
35. Drug delivery system according to clause 34, wherein one or more of the removable programming key comprises connectors configured to connect a plurality of programming keys to each other.
36. Drug delivery system according to clause 34 or 35, wherein each programming key comprises internal logic and/or memory configured to communicate with the drive unit and/or configured to control at least an aspect of drug delivery.
37. Drug delivery system according to any one of clauses 34 to 36, insofar as being dependent on clauses 1 to 18, wherein the drug unit interface is the removable programming key.
38. Drug delivery system according to any one of clauses 1 to 18 or 28 to 37, wherein the identification carries at least one of type of drug, temperature, flowrate, or sequence information,
   wherein the drive unit is configured to read the information carried by the identification.
39. Drug delivery system according to clause 38, wherein the drive unit is configured to provide, based on the read information, a virtual stack of drug units indicating a sequence of drug delivery.
40. Drug delivery system according to clause 39, wherein the drive unit comprises at least one indicator configured to provide visual feedback on the virtual stack to a user of the drug delivery system.
41. Drug delivery system according to clause 40, wherein the at least one indicator is one or more light emitting diodes, LEDs, and/or a display.
42. Drug delivery system according to clause 40 or 41, wherein the indicator is configured to output a warning or an error state relating to the virtual stack,
   wherein the warning or error state is output by emitting light, emitting light of different colours and/or emitting flashing light.
43. Drug delivery system according to any one of clauses 39 to 42, wherein the drive unit comprises at least one button,
   wherein the at least one button is configured to manage the virtual stack.
44. Drug delivery system according to clause 43, wherein managing the virtual stack comprises adding or deleting drug units and/or terminating addition or deletion of drug units and/or initiating drug delivery.
45. Drug delivery system according to any one of clauses 38 to 44, wherein the identification is a passive component,
   wherein the passive component is preferably an RFID chip or near field communication, NFC, chip.
46. Drug delivery system according to any one of clauses 35 to 37, wherein the connectors are configured to allow communication between or through the keys to the drive unit.

The delivery devices described herein can be used for the treatment and/or prophylaxis of one or more of many different types of disorders.

Exemplary disorders include, but are not limited to: rheumatoid arthritis, inflammatory bowel diseases (e.g. Crohn's disease and ulcerative colitis), hypercholesterolaemia and/or dyslipidemia, cardiovascular disease, diabetes (e.g. type 1 or 2 diabetes), psoriasis, psoriatic arthritis, spondyloarthritis, hidradenitis suppurativa, Sjögren's syndrome, migraine, cluster headache, multiple sclerosis, neuromyelitis optica spectrum disorder, anaemia, thalassemia, paroxysmal nocturnal hemoglobinuria, hemolytic anaemia, hereditary angioedema, systemic lupus erythematosus, lupus nephritis, myasthenia gravis, Behçet's disease, hemophagocytic lymphohistiocytosis, atopic dermatitis, retinal diseases (e.g., age-related macular degeneration, diabetic macular edema), uveitis, infectious diseases, bone diseases (e.g., osteoporosis, osteopenia), asthma, chronic obstructive pulmonary disease, thyroid eye disease, nasal polyps, transplant, acute hypoglycaemia, obesity, anaphylaxis, allergies, sickle cell disease, Alzheimer's disease, Parkinson's disease, dementia with Lewy bodies, systemic infusion reactions, immunoglobulin E (IgE)-mediated hypersensitivity reactions, cytokine release syndrome, immune deficiencies (e.g., primary immunodeficiency, chronic inflammatory demyelinating polyneuropathy), enzyme deficiencies (e.g., Pompe disease, Fabry disease, Gaucher disease), growth factor deficiencies, hormone deficiencies, coagulation disorders (e.g., hemophilia, von Willebrand disease, Factor V Leiden), and cancer.

Exemplary types of drugs that could be included in the delivery devices described herein include, but are not limited to, small molecules, hormones, cytokines, blood products, enzymes, vaccines, anticoagulants, immunosuppressants, antibodies, antibody-drug conjugates, neutralizing antibodies, reversal agents, radioligand therapies, radioisotopes and/or nuclear medicines, diagnostic agents, bispecific antibodies, proteins, fusion proteins, peptibodies, polypeptides, pegylated proteins, protein fragments, nucleotides, protein analogues, protein variants, protein precursors, protein derivatives, chimeric antigen receptor T cell therapies, cell or gene therapies, oncolytic viruses, or immunotherapies.

Exemplary drugs that could be included in the delivery devices described herein include, but are not limited to, immuno-oncology or bio-oncology medications such as immune checkpoints, cytokines, chemokines, clusters of differentiation, interleukins, integrins, growth factors, coagulation factors, enzymes, enzyme inhibitors, retinoids, steroids, signaling proteins, pro-apoptotic proteins, anti-apoptotic proteins, T-cell receptors, B-cell receptors, or costimulatory proteins.

Exemplary drugs that could be included in the delivery devices described herein include, but are not limited to, those exhibiting a proposed mechanism of action, such as human epidermal growth factor receptor 2 (HER-2) receptor modulators, interleukin (IL) modulators, interferon (IFN) modulators, complement modulators, glucagon-like peptide-1 (GLP-1) modulators, glucose-dependent insulinotropic polypeptide (GIP) modulators, cluster of differentiation 38 (CD38) modulators, cluster of differentiation 22 (CD22) modulators, C1 esterase modulators, bradykinin modulators, C-C chemokine receptor type 4 (CCR4) modulators, vascular endothelial growth factor (VEGF) modulators, B-cell activating factor (BAFF), P-selectin modulators, neonatal Fc receptor (FcRn) modulators, calcitonin gene-related peptide (CGRP) modulators, epidermal growth factor receptor (EGFR) modulators, cluster of differentiation 79B (CD79B) modulators, tumor-associated calcium signal transducer 2 (Trop-2) modulators, cluster of differentiation 52 (CD52) modulators, B-cell maturation antigen (BCMA) modulators, enzyme modulators, platelet-derived growth factor receptor A (PDGFRA) modulators, cluster of differentiation 319 (CD319 or SLAMF7) modulators, programmed cell death protein 1 and programmed death-ligand 1 (PD-1/PD-L1) inhibitors/modulators, B-lymphocyte antigen cluster of differentiation 19 (CD19) inhibitors, B-lymphocyte antigen cluster of differentiation 20 (CD20) modulators, cluster of differentiation 3 (CD3) modulators, cytotoxic T-lymphocyte-associated protein 4 (CTLA-4) inhibitors, T-cell immunoglobulin and mucin-domain containing-3 (TIM-3) modulators, T cell immunoreceptor with Ig and ITIM domains (TIGIT) modulators, V-domain Ig suppressor of T cell activation (VISTA) modulators, indoleamine 2,3-dioxygenase (IDO or INDO) modulators, poliovirus receptor-related immunoglobulin domain-containing protein (PVRIG) modulators, lymphocyte-activation gene 3 (LAG3; also known as cluster of differentiation 223 or CD223) antagonists, cluster of differentiation 276 (CD276 or B7-H3) antigen modulators, cluster of differentiation 47 (CD47) antagonists, cluster of differentiation 30 (CD30) modulators, cluster of differentiation 73 (CD73) modulators, cluster of differentiation 66 (CD66) modulators, cluster of differentiation W137 (CDW137) agonists, cluster of differentiation 158 (CD158) modulators, cluster of differentiation 27 (CD27) modulators, cluster of differentiation 58 (CD58) modulators, cluster of differentiation 80 (CD80) modulators, cluster of differentiation 33 (CD33) modulators, cluster of differentiation 159 (CD159 or NKG2) modulators, glucocorticoid-induced TNFR-related (GITR) protein modulators, Killer Ig-like receptor (KIR) modulators, growth arrest-specific protein 6 (GAS6)/AXL pathway modulators, A proliferation-inducing ligand (APRIL) receptor modulators, human leukocyte antigen (HLA) modulators, epidermal growth factor receptor (EGFR) modulators, B-lymphocyte cell adhesion molecule modulators, cluster of differentiation w123 (CDw123) modulators, Erbb2 tyrosine kinase receptor modulators, endoglin modulators, mucin modulators, mesothelin modulators, hepatitis A virus cellular receptor 2 (HAVCR2) antagonists, cancer-testis antigen (CTA) modulators, tumor necrosis factor receptor superfamily, member 4 (TNFRSF4 or OX40) modulators, adenosine receptor modulators, inducible T cell co-stimulator (ICOS) modulators, cluster of differentiation 40 (CD40) modulators, tumor-infiltrating lymphocytes (TIL) therapies, or T-cell receptor (TCR) therapies.

Exemplary drugs that could be included in the delivery devices described herein include, but are not limited to: etanercept, abatacept, adalimumab, evolocumab, exenatide, secukinumab, erenumab, galcanezumab, fremanezumab-vfrm, alirocumab, methotrexate (amethopterin), tocilizumab, interferon beta-1a, interferon beta-1b, peginterferon beta-1a, sumatriptan, darbepoetin alfa, belimumab, sarilumab, semaglutide, dupilumab, reslizumab, omalizumab, glucagon, epinephrine, naloxone, insulin, amylin, vedolizumab, eculizumab, ravulizumab, crizanlizumab-tmca, certolizumab pegol, satralizumab, denosumab, romosozumab, benralizumab, emicizumab, tildrakizumab, ocrelizumab, ofatumumab, natalizumab, mepolizumab, risankizumab-rzaa, ixekizumab, and immune globulins.

Exemplary drugs that could be included in the delivery devices described herein may also include, but are not limited to, oncology treatments such as ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab.

Exemplary drugs that could be included in the delivery devices described herein include “generic” or biosimilar equivalents of any of the foregoing, and the foregoing molecular names should not be construed as limiting to the “innovator” or “branded” version of each, as in the non-limiting example of innovator medicament adalimumab and biosimilars such as adalimumab-afzb, adalimumab-atto, adalimumab-adbm, and adalimumab-adaz.

Exemplary drugs that could be included in the delivery devices described herein also include, but are not limited to, those used for adjuvant or neoadjuvant chemotherapy, such as an alkylating agent, plant alkaloid, antitumor antibiotic, antimetabolite, or topoisomerase inhibitor, enzyme, retinoid, or corticosteroid. Exemplary chemotherapy drugs include, by way of example but not limitation, 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine.

Exemplary drugs that could be included in the delivery devices described herein also include, but are not limited to, analgesics (e.g., acetaminophen), antipyretics, corticosteroids (e.g. hydrocortisone, dexamethasone, or methylprednisolone), antihistamines (e.g., diphenhydramine or famotidine), antiemetics (e.g., ondansetron), antibiotics, antiseptics, anticoagulants, fibrinolytics (e.g., recombinant tissue plasminogen activator [r-TPA]), antithrombolytics, or diluents such as sterile water for injection (SWFI), 0.9% Normal Saline, 0.45% normal saline, 5% dextrose in water, 5% dextrose in 0.45% normal saline, Lactated Ringer's solution, Heparin Lock Flush solution, 100 U/mL Heparin Lock Flush Solution, or 5000 U/mL Heparin Lock Flush Solution.

Pharmaceutical formulations including, but not limited to, any drug described herein are also contemplated for use in the delivery devices described herein, for example pharmaceutical formulations comprising a drug as listed herein (or a pharmaceutically acceptable salt of the drug) and a pharmaceutically acceptable carrier. Such formulations may include one or more other active ingredients (e.g., as a combination of one or more active drugs), or may be the only active ingredient present, and may also include separately administered or co-formulated dispersion enhancers (e.g. an animal-derived, human-derived, or recombinant hyaluronidase enzyme), concentration modifiers or enhancers, stabilizers, buffers, or other excipients.

Exemplary drugs that could be included in the delivery devices described herein include, but are not limited to, a multi-medication treatment regimen such as AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, Mini-CHOP, Maxi-CHOP, VR-CAP, Dose-Dense CHOP, EPOCH, Dose-Adjusted EPOCH, R-EPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOPAD, 7 +3, 5 +2, 7+4, MEC, CVP, RBAC500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DT-PACE, VTD-PACE, DCEP, ATG, VAC, VeIP, OFF, GTX, CAV, AD, MAID, AIM, VAC-IE, ADOC, or PE.

1-15. (canceled)

16. A drug delivery system comprising:
a drive unit,
wherein the drive unit comprises a drive unit interface, a drug interface, a pump assembly or a motor assembly configured to deliver a drug, a drug delivery controller and a power source;
at least one drug unit connectable to the drive unit,
wherein the drug unit comprises a drug reservoir for storing a drug, an identification comprising information on the drug, a drug interface communicating with the drug reservoir;
a drug unit interface,
wherein the drug unit interface is configured to communicate with the drive unit interface,
wherein the drug interface of the drug unit is configured to communicate with the drive interface of the drive unit; and
a terminator unit connectable to the drug unit,
wherein the terminator unit comprises one or more components of a system controller, a terminator unit interface configured to communicate with the drug unit interface, and
wherein the terminator unit is configured to close at least one of the drug unit interface and the drug interface of the drug unit.

17. The drug delivery system according to claim 16, wherein the drive unit interface and the drug unit interface comprise at least one of a mechanical or electrical interface.

18. The drug delivery system according to claim 16, wherein the drug interface of the drive unit and the drug interface of the drug unit comprise at least one of a mechanical connection, a sealed pneumatic connection, or an electrical connection.

19. The drug delivery system according to claim 16, wherein the system comprises a plurality of drug units,
wherein each of the plurality of drug units comprises identical drug interfaces and drug unit interfaces,
wherein the drug units are configured to be connected via the drug interface and the drug unit interface,
wherein the drug units are configured to be connected between the drive unit and the terminator unit.

20. The drug delivery system according to claim 19, wherein the drug units are configured to be connected between the drive unit and the terminator unit to form a stack, and/or
wherein the drug units are configured to be connected between the drive unit and the terminator unit in an order corresponding to a planned order of administration to a patient.

21. The drug delivery system according to claim 16, wherein the units are configured to communicate via a wired or wireless connection, preferably via the electrical interfaces.

22. The drug delivery system according to claim 16, wherein the drug delivery controller is configured to control the pump assembly or motor assembly for delivering the drug to a patient, and
wherein the drug delivery controller preferably comprises a non-volatile memory.

23. The drug delivery system according to claim 16, wherein at least one of the drive unit, the drug unit or the terminator unit comprises at least one user interface element configured to control, indicate, or control and indicate a system status.

24. The drug delivery system according to claim 23,
wherein the at least one interface element is at least one of a button, LED or screen.

25. The drug delivery system according to claim 23,
wherein the system status indicates at least one of a correct connection of the units, sequence of the units, information on expiry of the drug, battery level, dispense parameter information, dispense error information, dispense readiness information, dispense status information, connection status, patient clinical characteristics, occlusion status of a tubing set, or needle used with the system.

26. The drug delivery system according to claim 16, wherein at least one of the drug unit or the terminator unit comprises a wireless or wired connection module configured to communicate with at least one of a server, the internet or a terminal.

27. The drug delivery system according to claim 26,
wherein the wireless or wired connection module is configured to receive and transmit drug delivery information,
wherein preferably, the drug delivery information is received or transmitted during filling of the drug reservoir within the drug unit, and/or
wherein preferably, the drug delivery information corresponds to one or more aspects contained in an electronic health record system for a patient using the drug delivery system.

28. The drug delivery system according to claim 16, wherein the drug unit further comprises a temperature sensor configured to sense at least one of a temperature of a drug stored in the drug reservoir or an ambient temperature where the drug delivery system is used.

29. The drug delivery system according to claim 16, wherein the identification is stored in an electronic memory, an RFID tag, a 2D barcode, a data matrix, or a QR code.

30. The drug delivery system according to claim 29,
wherein the identification comprises information on at least one of a medication contained a drug unit, a volume of the drug reservoir, volume of medication within the drug reservoir, drug delivery information, dispense parameters of the drug, flow rate of the drug, drug lot number, patient identifier, an identifier of a medication in a sequence of one or more medications, expiration date, a temperature condition of the drug, administration date, administration time, medication cycle number, serialization/identifier information for one or more of the medication, or drug unit.
